(19) United States
(12) Patent Application Publication
(10) Pub. No.: US 2011/0079773 A1
(43) Pub. Date: Apr. 7, 2011

(54) Selectively Functionalized Rylene Imides and Diimides

(76) Inventors: Michael R. Wasielewski, Glenview, IL (US); Joseph E. Bullock, Chicago, IL (US); Michael T. Vagnini, Evanston, IL (US)

(21) Appl. No.: 12/860,574

(22) Filed: Aug. 20, 2010

Related U.S. Application Data
(60) Provisional application No. 61/235,832, filed on Aug. 21, 2009.

(51) Int. Cl. H01L 51/30; C07D 471/04; C07D 221/06; H01L 51/46; H01L 51/54

(52) U.S. Cl. 257/40; 546/37; 546/26; 546/98; 257/E51.027

(57) ABSTRACT

Disclosed are new selectively functionalized rylene imides and diimides that can exhibit desirable electronic properties and can possess processing advantages including solution-processability and/or good stability at ambient conditions.

### CROSS REFERENCE TO RELATED APPLICATIONS

This application claims priority to and the benefit of U.S. Provisional Patent Application Ser. No. 61/235,832, filed on Aug. 21, 2009, the disclosure of which is incorporated by reference herein in its entirety.

### STATEMENT OF GOVERNMENT INTEREST

This invention was made with government support under Grant No. DE-FG02-99ER14999 awarded by the U.S. Department of Energy, Office of Basic Energy Sciences. The government has certain rights in the invention.

### BACKGROUND

For decades, rylene imide compounds have been intensively studied as useful light absorbers, fluorescent tags, and as electron donors/acceptors. The naphthalene derivatives, naphthalene-1,8-dicarboximides (NMI) and naphthalene-1,4:5,8-bis(dicarboximides) (NI), have been used in artificial photosynthetic systems, while the higher homologues, perylene-3,4-dicarboximides (PMI), perylene-3, 4:9,10-bis(dicarboximides) (PDI), and terrylene-3,4:11,12-bis(dicarboximides) (TDI) have been extensively researched not only in artificial photosynthetic systems, but also in light harvesting systems and solid state devices such as organic field-effect transistors (OFETs) and photovoltaics. In all these systems, chemical functionalization through the imide has been shown to proceed efficiently starting from either the dicarboxylic acid or the cyclic anhydride. Molecular orbital calculations on these systems show that they all exhibit a nodal plane in both their HOMO and LUMO that bisects the molecules through their imide nitrogen(s), which decouples the imide substituent from the electronic structure of the rylene imide or diimide chromophore. Thus, a diverse array of alkyl and aryl imide groups have been prepared having negligible impact on the optical and electrochemical properties of the chromophores.

While the imide groups can confer some solubility to the compound, larger derivatives such as perylene and terrylene derivatives often are insoluble in common solvents and require additional solubilizing groups attached to the aromatic core. Under most existing methods, such core substitutions typically require bromination or chlorination of the aromatic core, and are performed under highly acidic conditions. In addition, because halogenation of PMI, PDI, and TDI generally takes place in the bay-region of the aromatic core, substitution with additional solubilizing groups is limited to the bay-region of the aromatic core. However, the steric effects of bay substitution can disrupt the planarity of the aromatic core, which can affect the vibronic structure adversely. To minimize this effect, synthetic strategies need to avoid substitution in the bay region and instead provide access to other positions, for example, positions ortho to the imide groups.

Accordingly, there is a need in the art for rylene imides and rylene diimides that are selectively functionalized at positions ortho to the imide groups. Such substitutions would be beneficial for solubility while maintaining the optical properties of the parent chromophore.

### SUMMARY

In light of the foregoing, the present teachings provide various compounds that can address certain deficiencies and shortcomings of the prior art, including those outlined above. More specifically, the present teachings relate to compounds having the formula:

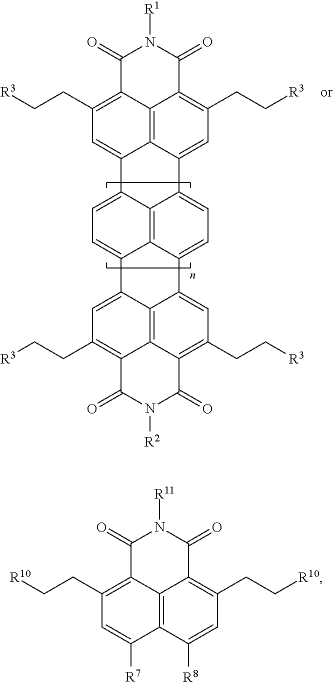

where R¹, R², R³, R⁷, R⁸, R¹⁰, R¹¹, and n are as defined herein. Also provided are associated devices and related methods for the preparation and use of these compounds.

The foregoing as well as other features and advantages of the present teachings will be more fully understood from the following figures, description, examples, and claims.

### BRIEF DESCRIPTION OF DRAWINGS

It should be understood that the drawings described below are for illustration purpose only. The drawings are not necessarily to scale, with emphasis generally being placed upon illustrating the principles of the present teachings. The drawings are not intended to limit the scope of the present teachings in any way.

FIGS. 1a-d illustrate four different configurations of thin film transistors: bottom-gate top contact (FIG. 1a), bottom-gate bottom-contact (FIG. 1b), top-gate bottom-contact (FIG. 1c), and top-gate top-contact (FIG. 1d); each of which can be used to incorporate compounds of the present teachings.

FIG. 2 illustrates a representative structure of a bulk-heterojunction organic photovoltaic device (also known as solar cell) which can incorporate one or more compounds of the present teachings as the donor and/or acceptor materials.

FIG. 3 illustrates a representative structure of an organic light-emitting device which can incorporate one or more compounds of the present teachings as electron-transporting and/or emissive and/or hole-transporting materials.

FIGS. 4a and 4b provide representative UV-vis absorption and fluorescence spectra of certain compounds according to the present teachings. FIG. 4a shows the UV-vis absorption of compounds 2, 4, 6, 8, and 10 in toluene. FIG. 4b shows the corresponding fluorescence spectra.

FIG. 5 shows the electron paramagnetic resonance (EPR) spectra of compound 8^{•-} in dichloromethane (DCM) with ˜2% triethyamine (TEA) at 290 K. Microwave power was 2 mW with a modulation amplitude of 0.1 G at 25 kHz. The simulation is constrained by electron nuclear double resonance (ENDOR) data for protons, and the nitrogen hyperfine coupling constant (hfcc) is optimized.

FIG. 6 shows the ¹H-ENDOR spectra of compound 8^{•-} in DCM with ˜2% TEA at 290 K. Microwave power was 6 mW and RF power was 240-400 W with a frequency modulation depth of 50 kHz.

### DETAILED DESCRIPTION

The present teachings provide various rylene imides and diimides that are functionalized at positions ortho to the imide groups, as well as compositions, composites, and/or devices associated with these compounds. Compared to otherwise similar but unsubstituted rylene imides and diimides, the present compounds can exhibit significantly higher solubility. In addition, unlike rylene imides and diimides that are substituted at the bay region of the aromatic core, the present compounds do not have a twisted molecular structure and accordingly, can have better electronic properties. For example, the present compounds can exhibit semiconductor behavior such as high carrier mobility and/or good current modulation characteristics in a field-effect device, light absorption/charge separation in a photovoltaic device, and/or charge transport/recombination/light emission in a light-emitting device. Furthermore, the present compounds can possess certain processing advantages such as good stability (for example, air stability) in ambient conditions. The compounds of the present teachings can be used to prepare either p-type or n-type semiconductor materials, which in turn can be used to fabricate various organic electronic articles, structures and devices, including field-effect transistors, unipolar circuitries, complementary circuitries, photovoltaic devices, and light emitting devices.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components or can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, a “cyclic moiety” can include one or more (e.g., 1-6) carbocyclic or heterocyclic rings. The cyclic moiety can be a cycloalkyl group, a heterocycloalkyl group, an aryl group, or a heteroaryl group (i.e., can include only saturated bonds, or can include one or more unsaturated bonds regardless of aromaticity), each including, for example, 3-24 ring atoms and can be optionally substituted as described herein. In embodiments where the cyclic moiety is a “monocyclic moiety,” the “monocyclic moiety” can include a 3-14 membered aromatic or non-aromatic, carbocyclic or heterocyclic ring. A monocyclic moiety can include, for example, a phenyl group or a 5- or 6-membered heteroaryl group, each of which can be optionally substituted as described herein. In embodiments where the cyclic moiety is a “polycyclic moiety,” the “polycyclic moiety” can include two or more rings fused to each other (i.e., sharing a common bond) and/or connected to each other via a spiro atom, or one or more bridged atoms. A polycyclic moiety can include an 8-24 membered aromatic or non-aromatic, carbocyclic or heterocyclic ring, such as a C₈₋₂₄ aryl group or an 8-24 membered heteroaryl group, each of which can be optionally substituted as described herein.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. Examples of alkyl groups include methyl (Me), ethyl (Et), propyl (e.g., n-propyl and iso-propyl), butyl (e.g., n-butyl, iso-butyl, sec-butyl, tert-butyl), pentyl groups (e.g., n-pentyl, iso-pentyl, neopentyl), hexyl groups, and the like. In various embodiments, an alkyl group can have 1 to 40 carbon atoms (i.e., C₁₋₄₀ alkyl group), for example, 1-20 carbon atoms (i.e., C₁₋₂₀ alkyl group). In some embodiments, an alkyl group can have 1 to 6 carbon atoms, and can be referred to as a “lower alkyl group.” Examples of lower alkyl groups include methyl, ethyl, propyl (e.g., n-propyl and iso-propyl), and butyl groups (e.g., n-butyl, iso-butyl, sec-butyl, tert-butyl). In some embodiments, alkyl groups can be substituted as described herein. An alkyl group is generally not substituted with another alkyl group, an alkenyl group, or an alkynyl group.

As used herein, “haloalkyl” refers to an alkyl group having one or more halogen substituents. At various embodiments, a haloalkyl group can have 1 to 40 carbon atoms (i.e., C₁₋₄₀ haloalkyl group), for example, 1 to 20 carbon atoms (i.e., C₁₋₂₀ haloalkyl group). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups where all of the hydrogen atoms are replaced with halogen atoms (e.g., CF₃ and C₂F₅), are included within the definition of “haloalkyl.” For example, a C₁₋₄₀ haloalkyl group can have the formula —C_{z}H_{2z+1-t}X⁰ₜ, where X⁰, at each occurrence, is F, Cl, Br or I, z is an integer in the range of 1 to 40, and t is an integer in the range of 1 to 81, provided that t is less than or equal to 2z+1. Haloalkyl groups that are not perhaloalkyl groups can be substituted as described herein.

As used herein, “alkoxy” refers to —O-alkyl group. Examples of alkoxy groups include, but are not limited to, methoxy, ethoxy, propoxy (e.g., n-propoxy and isopropoxy), t-butoxy, pentoxyl, hexoxyl groups, and the like. The alkyl group in the —O-alkyl group can be substituted as described herein.

As used herein, “alkylthio” refers to an —S-alkyl group (which, in some cases, can be expressed as —S(O)_{w}-alkyl, wherein w is 0). Examples of alkylthio groups include, but are not limited to, methylthio, ethylthio, propylthio (e.g., n-propylthio and isopropylthio), t-butylthio, pentylthio, hexylthio groups, and the like. The alkyl group in the —S-alkyl group can be substituted as described herein.

As used herein, “arylalkyl” refers to an -alkyl-aryl group, where the arylalkyl group is covalently linked to the defined chemical structure via the alkyl group. An arylalkyl group is within the definition of a —Y—C₆₋₁₄ aryl group, where Y is as defined herein. An example of an arylalkyl group is a benzyl group (—CH₂—C₆H₅). An arylalkyl group can be optionally substituted, i.e., the aryl group and/or the alkyl group, can be substituted as disclosed herein.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C₂₋₄₀ alkenyl group), for example, 2 to 20 carbon atoms (i.e., C₂₋₂₀ alkenyl group). In some embodiments, alkenyl groups can be substituted as described herein. An alkenyl group is generally not substituted with another alkenyl group, an alkyl group, or an alkynyl group.

As used herein, “alkynyl” refers to a straight-chain or branched alkyl group having one or more triple carbon-carbon bonds. Examples of alkynyl groups include ethynyl, propynyl, butynyl, pentynyl, hexynyl, and the like. The one or more triple carbon-carbon bonds can be internal (such as in 2-butyne) or terminal (such as in 1-butyne). In various embodiments, an alkynyl group can have 2 to 40 carbon atoms (i.e., C₂₋₄₀ alkynyl group), for example, 2 to 20 carbon atoms (i.e., C₂₋₂₀ alkynyl group). In some embodiments, alkynyl groups can be substituted as described herein. An alkynyl group is generally not substituted with another alkynyl group, an alkyl group, or an alkenyl group.

As used herein, “cycloalkyl” refers to a non-aromatic carbocyclic group including cyclized alkyl, alkenyl, and alkynyl groups. In various embodiments, a cycloalkyl group can have 3 to 24 carbon atoms, for example, 3 to 20 carbon atoms (e.g., C₃₋₁₄ cycloalkyl group). A cycloalkyl group can be monocyclic (e.g., cyclohexyl) or polycyclic (e.g., containing fused, bridged, and/or spiro ring systems), where the carbon atoms are located inside or outside of the ring system. Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted as described herein.

As used herein, “heteroatom” refers to an atom of any element other than carbon or hydrogen and includes, for example, nitrogen, oxygen, silicon, sulfur, phosphorus, and selenium.

As used herein, “cycloheteroalkyl” refers to a non-aromatic cycloalkyl group that contains at least one ring heteroatom selected from O, S, Se, N, P, and Si (e.g., O, S, and N), and optionally contains one or more double or triple bonds. A cycloheteroalkyl group can have 3 to 24 ring atoms, for example, 3 to 20 ring atoms (e.g., 3-14 membered cycloheteroalkyl group). One or more N, P, S, or Se atoms (e.g., N or S) in a cycloheteroalkyl ring may be oxidized (e.g., morpholine N-oxide, thiomorpholine S-oxide, thiomorpholine S,S-dioxide). In some embodiments, nitrogen or phosphorus atoms of cycloheteroalkyl groups can bear a substituent, for example, a hydrogen atom, an alkyl group, or other substituents as described herein. Cycloheteroalkyl groups can also contain one or more oxo groups, such as oxopiperidyl, oxooxazolidyl, dioxo-(1H,3H)-pyrimidyl, oxo-2(1H)-pyridyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like. In some embodiments, cycloheteroalkyl groups can be substituted as described herein.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have 6 to 24 carbon atoms in its ring system (e.g., C₆₋₂₀ aryl group), which can include multiple fused rings. In some embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, aryl groups can be substituted as described herein. In some embodiments, an aryl group can have one or more halogen substituents, and can be referred to as a “haloaryl” group. Perhaloaryl groups, i.e., aryl groups where all of the hydrogen atoms are replaced with halogen atoms (e.g., —C₆F₅), are included within the definition of “haloaryl.” In certain embodiments, an aryl group is substituted with another aryl group and can be referred to as a biaryl group. Each of the aryl groups in the biaryl group can be substituted as disclosed herein.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5- or 6-membered monocyclic and 5-6 bicyclic ring systems shown below:

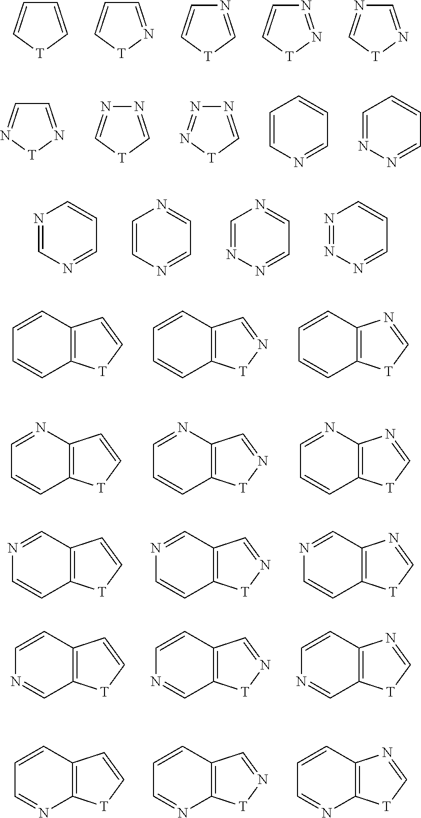

where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted as described herein.

Compounds of the present teachings can include a “divalent group” defined herein as a linking group capable of forming a covalent bond with two other moieties. For example, compounds of the present teachings can include a divalent C₁₋₄₀ alkyl group (e.g., a methylene group), a divalent C₂₋₄₀ alkenyl group (e.g., a vinylyl group), a divalent C₂₋₄₀ alkynyl group (e.g., an ethynylyl group). a divalent C₆₋₁₄ aryl group (e.g., a phenylyl group); a divalent 3-14 membered cycloheteroalkyl group (e.g., a pyrrolidylyl), and/or a divalent 5-14 membered heteroaryl group (e.g., a thienylyl group). Generally, a chemical group (e.g., —Ar—) is understood to be divalent by the inclusion of the two bonds before and after the group.

The electron-donating or electron-withdrawing properties of several hundred of the most common substituents, reflecting all common classes of substituents have been determined, quantified, and published. The most common quantification of electron-donating and electron-withdrawing properties is in terms of Hammett σ values. Hydrogen has a Hammett σ value of zero, while other substituents have Hammett σ values that increase positively or negatively in direct relation to their electron-withdrawing or electron-donating characteristics. Substituents with negative Hammett σ values are considered electron-donating, while those with positive Hammett σ values are considered electron-withdrawing. See Lange's Handbook of Chemistry, 12th ed., McGraw Hill, 1979, Table 3-12, pp. 3-134 to 3-138, which lists Hammett σ values for a large number of commonly encountered substituents and is incorporated by reference herein.

It should be understood that the term “electron-accepting group” can be used synonymously herein with “electron acceptor” and “electron-withdrawing group”. In particular, an “electron-withdrawing group” (“EWG”) or an “electron-accepting group” or an “electron-acceptor” refers to a functional group that draws electrons to itself more than a hydrogen atom would if it occupied the same position in a molecule. Examples of electron-withdrawing groups include, but are not limited to, halogen or halo (e.g., F, Cl, Br, I), —NO₂, —CN, —NC, —S(R⁰)₂⁺, —N(R⁰)₃⁺, —SO₃H, —SO₂R⁰, —SO₃R⁰, —SO₂NHR⁰, —SO₂N(R⁰)₂, —COOH, —COR⁰, —COOR⁰, —CONHR⁰, —CON(R⁰)₂, C₁₋₄₀ haloalkyl groups, C₆₋₁₄ aryl groups, and 5-14 membered electron-poor heteroaryl groups; where R⁰ is a C₁₋₄₀ alkyl group, a C₂₋₄₀ alkenyl group, a C₂₋₄₀ alkynyl group, a C₁₋₄₀ haloalkyl group, a C₁₋₄₀ alkoxy group, a C₆₋₁₄ aryl group, a C₃₋₁₄ cycloalkyl group, a 3-14 membered cycloheteroalkyl group, and a 5-14 membered heteroaryl group, each of which can be optionally substituted as described herein.

It should be understood that the term “electron-donating group” can be used synonymously herein with “electron donor”. In particular, an “electron-donating group” or an “electron-donor” refers to a functional group that donates electrons to a neighboring atom more than a hydrogen atom would if it occupied the same position in a molecule. Examples of electron-donating groups include —OH, —OR⁰, —NH₂, —NHR⁰, —N(R⁰)₂, 5-14 membered electron-rich heteroaryl groups, C₁₋₄₀ alkyl groups, C₂₋₄₀ alkenyl groups, C₂₋₄₀ alkynyl groups, C₁₋₄₀ alkoxy groups, where R⁰ is a C₁₋₄₀ alkyl group, a C₂₋₄₀ alkenyl group, a C₂₋₄₀ alkynyl group, a C₆₋₁₄ aryl group, or a C₃₋₁₄ cycloalkyl group.

Various unsubstituted heteroaryl groups can be described as electron-rich (or π-excessive) or electron-poor (or π-deficient). Such classification is based on the average electron density on each ring atom as compared to that of a carbon atom in benzene. Examples of electron-rich systems include 5-membered heteroaryl groups having one heteroatom such as furan, pyrrole, and thiophene; and their benzofused counterparts such as benzofuran, benzopyrrole, and benzothiophene. Examples of electron-poor systems include 6-membered heteroaryl groups having one or more heteroatoms such as pyridine, pyrazine, pyridazine, and pyrimidine; as well as their benzofused counterparts such as quinoline, isoquinoline, quinoxaline, cinnoline, phthalazine, naphthyridine, quinazoline, phenanthridine, acridine, and purine. Mixed heteroaromatic rings can belong to either class depending on the type, number, and position of the one or more heteroatom(s) in the ring. See Katritzky, A. R and Lagowski, J. M., Heterocyclic Chemistry (John Wiley & Sons, New York, 1960).

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

Compounds described herein can contain an asymmetric atom (also referred as a chiral center) and some of the compounds can contain two or more asymmetric atoms or centers, which can thus give rise to optical isomers (enantiomers) and diastereomers (geometric isomers). The present teachings include such optical isomers and diastereomers, including their respective resolved enantiomerically or diastereomerically pure isomers (e.g., (+) or (−) stereoisomer) and their racemic mixtures, as well as other mixtures of the enantiomers and diastereomers. In some embodiments, optical isomers can be obtained in enantiomerically enriched or pure form by standard procedures known to those skilled in the art, which include, for example, chiral separation, diastereomeric salt formation, kinetic resolution, and asymmetric synthesis. The present teachings also encompass cis- and trans-isomers of compounds containing alkenyl moieties (e.g., alkenes, azo, and imines). It also should be understood that the compounds of the present teachings encompass all possible regioisomers in pure form and mixtures thereof. In some embodiments, the preparation of the present compounds can include separating such isomers using standard separation procedures known to those skilled in the art, for example, by using one or more of column chromatography, thin-layer chromatography, simulated moving-bed chromatography, and high-performance liquid chromatography. However, mixtures of regioisomers can be used similarly to the uses of each individual regioisomer of the present teachings as described herein and/or known by a skilled artisan.

It is specifically contemplated that the depiction of one regioisomer includes any other regioisomers and any regioisomeric mixtures unless specifically stated otherwise.

As used herein, a “leaving group” (“LG”) refers to a charged or uncharged atom (or group of atoms) that can be displaced as a stable species as a result of, for example, a substitution or elimination reaction. Examples of leaving groups include, but are not limited to, halogen (e.g., Cl, Br, I), azide (N₃), thiocyanate (SCN), nitro (NO₂), cyanate (CN), water (H₂O), ammonia (NH₃), and sulfonate groups (e.g., OSO₂—R, wherein R can be a C₁₋₁₀ alkyl group or a C₆₋₁₄ aryl group each optionally substituted with 1-4 groups independently selected from a C₁₋₁₀ alkyl group and an electron-withdrawing group) such as tosylate (toluenesulfonate, OTs), mesylate (methanesulfonate, OMs), brosylate (p-bromobenzenesulfonate, OBs), nosylate (4-nitrobenzenesulfonate, ONs), and triflate (trifluoromethanesulfonate, OTf).

As used herein, a “p-type semiconductor material” or a “p-type semiconductor” refers to a semiconductor material having holes as the majority current carriers. In some embodiments, when a p-type semiconductor material is deposited on a substrate, it can provide a hole mobility in excess of about 10⁻⁵ cm²/Vs. In the case of field-effect devices, a p-type semiconductor can also exhibit a current on/off ratio of greater than about 10.

As used herein, an “n-type semiconductor material” or an “n-type semiconductor” refers to a semiconductor material having electrons as the majority current carriers. In some embodiments, when an n-type semiconductor material is deposited on a substrate, it can provide an electron mobility in excess of about 10⁻⁵ cm²/Vs. In the case of field-effect devices, an n-type semiconductor can also exhibit a current on/off ratio of greater than about 10.

As used herein, “field effect mobility” refers to a measure of the velocity with which charge carriers, for example, holes (or units of positive charge) in the case of a p-type semiconductor material and electrons in the case of an n-type semiconductor material, move through the material under the influence of an electric field.

As used herein, a compound can be considered “ambient stable” or “stable at ambient conditions” when a transistor incorporating the compound as its semiconducting material exhibits a carrier mobility that is maintained at about its initial measurement when the compound is exposed to ambient conditions, for example, air, ambient temperature, and humidity, over a period of time. For example, a compound can be described as ambient stable if a transistor incorporating the compound shows a carrier mobility that does not vary more than 20% or more than 10% from its initial value after exposure to ambient conditions, including, air, humidity and temperature, over a 3 day, 5 day, or 10 day period.

As used herein, “solution-processable” refers to compounds, materials, or compositions that can be used in various solution-phase processes including spin-coating, printing (e.g., inkjet printing, screen printing, pad printing, offset printing, gravure printing, flexographic printing, lithographic printing, mass-printing and the like), spray coating, electrospray coating, drop casting, dip coating, and blade coating.

Throughout the specification, structures may or may not be presented with chemical names. Where any question arises as to nomenclature, the structure prevails.

In one aspect, the present teachings relate to compounds of formula I:

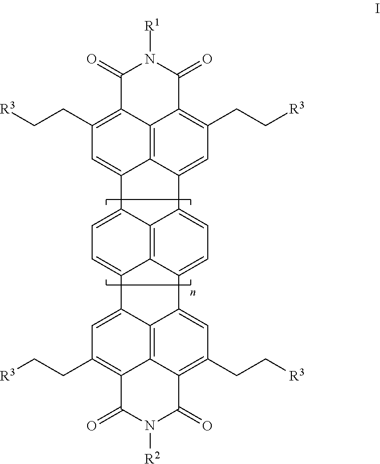

wherein:
R¹ and R² independently are selected from H, a C₁₋₄₀ alkyl group, a C₂₋₄₀ alkenyl group, a C₁₋₄₀ haloalkyl group, and an organic group comprising 1-4 cyclic moieties,
wherein:
each of the C₁₋₄₀ alkyl group, the C₂₋₄₀ alkenyl group, and the C₁₋₄₀ haloalkyl group optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group can be bonded covalently to the imide nitrogen atom via an optical linker; and
each of the 1-4 cyclic moieties in the organic group can be the same or different, can be bonded covalently to each other via an optical linker, and optionally can be substituted with 1-5 substitutents independently selected from a halogen, oxo, —CN, NO₂, OH, ═C(CN)₂, —NH₂, —NH(C₁₋₄₀ alkyl), —N(C₁₋₄₀ alkyl)₂, —S(O)₂OH, —CHO, —C(O)OH, —C(O)—C₁₋₄₀ alkyl, -sC(O)—OC₁₋₄₀ alkyl, —C(O)NH₂, —C(O)NH—C₁₋₄₀ alkyl, —C(O)N(C₁₋₄₀ alkyl)₂, —SiH₃, —SiH(C₁₋₄₀ alkyl)₂, —SiH₂(C₁₋₄₀ alkyl), —Si(C₁₋₄₀ alkyl)₃, —O—C₁₋₄₀ alkyl, —O—C₁₋₄₀ alkenyl, —O—C₁₋₄₀ haloalkyl, a C₁₋₄₀ alkyl group, a C₁₋₄₀ alkenyl group, and a C₁₋₄₀ haloalkyl group;
   R³ is selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 cycloheteroalkyl group optionally can be substituted with 1-10 substitutents independently selected from a halogen, —CN, a C₁₋₁₀ alkyl group, a C₁₋₁₀ alkoxy group, and a C₁₋₁₀ haloalkyl group; and
   n is 0, 1, or 2.

In certain embodiments, n can be 0. Compounds of formula I according to these embodiments can be represented by:

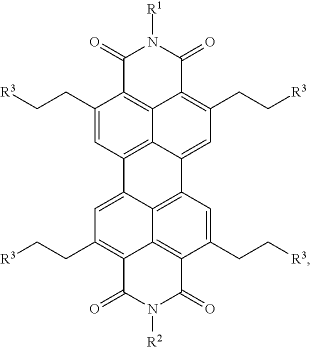

that is, perylene-3, 4:9,10-bis(dicarboximides) (or simply, perylene diimides) that are substituted with four —(CH₂)₂R³ groups at positions ortho to the imide groups, where R¹, R², and R³ are as defined herein.

In certain embodiments, n can be 1. Compounds of formula I according to these embodiments can be represented by:

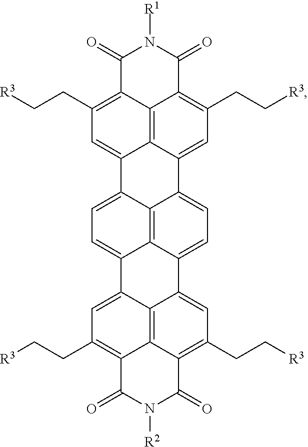

that is, terrylene-3,4:11,12-bis(dicarboximides) (or simply, terrylene diimides) that are substituted with four —(CH₂)₂R³ groups at positions ortho to the imide groups, where R¹, R², and R³ are as defined herein.

In various embodiments, R¹ and R² independently can be H or a substituent as described herein. For example, in various embodiments, R¹ and R² independently can be H or a substituent which can impart improved desirable properties to the compound as a whole. For example, certain substituents including one or more electron-withdrawing or electron-donating moieties can modulate the electronic properties of the compound, while substituents that include one or more aliphatic chains can improve the solubility of the compound in organic solvents.

Accordingly, in certain embodiments, R¹ and R² independently can be a linear or branched C₃₋₄₀ alkyl group, examples of which include an n-hexyl group, an n-octyl group, an n-dodecyl group, a 1-methylpropyl group, a 1-methylbutyl group, a 1-methylpentyl group, a 1-methylhexyl group, a 1-ethylpropyl group, a 1-ethylbutyl group, a 1,3-dimethylbutyl group, a 2-ethylhexyl group, a 2-hexyloctyl group, a 2-octyldodecyl group, and a 2-decyltetradecyl group. In certain embodiments, R¹ and R² independently can be a linear or branched C₃₋₄₀ alkenyl group (such as the linear or branched C₃₋₄₀ alkyl groups specified above but with one or more saturated bonds replaced by unsaturated bonds). In particular embodiments, R¹ and R² independently can be a branched C₃₋₂₀ alkyl group or a branched C₃₋₂₀ alkenyl group.

In certain embodiments, R¹ and R² independently can be a linear or branched C₆₋₄₀ alkyl or alkenyl group, an arylalkyl group (e.g., a benzyl group) substituted with a linear or branched C₆₋₄₀ alkyl or alkenyl group, an aryl group (e.g., a phenyl group) substituted with a linear or branched C₆₋₄₀ alkyl or alkenyl group, or a biaryl group (e.g., a biphenyl group) substituted with a linear or branched C₆₋₄₀ alkyl or alkenyl group, wherein each of these groups optionally can be substituted with 1-5 halo groups (e.g., F). In some embodiments, R¹ and R² independently can be a biaryl group wherein the two aryl groups are covalently linked via a linker. For example, the linker can be a divalent C₁₋₄₀ alkyl group wherein one or more non-adjacent CH₂ groups optionally can be replaced by —O—, —S—, or —Se—, i.e., O, S, and/or Se atoms are not linked directly to one another. The linker can include other heteroatoms and/or functional groups as described herein.

More generally, R¹ and R² independently can be selected from H, a C₁₋₄₀ alkyl group, a C₂₋₄₀ alkenyl group, a C₁₋₄₀ haloalkyl group, and an organic group comprising 1-4 cyclic moieties, wherein:
each of the C₁₋₄₀ alkyl group, the C₂₋₄₀ alkenyl group, and the C₁₋₄₀ haloalkyl group optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group can be covalently bonded to the imide nitrogen atom via an optional linker; and
each of the 1-4 cyclic moieties in the organic group can be the same or different, can be bonded covalently to each other or the imide nitrogen via an optional linker, and optionally can be substituted with 1-5 substituents independently selected from a halogen, —CN, oxo, NO₂, OH, ═C(CN)₂, —NH₂, —NH(C₁₋₄₀ alkyl), —N(C₁₋₄₀ alkyl)₂, —S(O)₂OH, —CHO, —C(O)OH, —C(O)—C₁₋₄₀ alkyl, —C(O)—OC₁₋₄₀ alkyl, —C(O)NH₂, —C(O)NH—C₁₋₄₀ alkyl, —C(O)N(C₁₋₄₀ alkyl)₂, —SiH₃, —SiH(C₁₋₄₀ alkyl)₂, —SiH₂(C₁₋₄₀ alkyl), —Si(C₁₋₄₀ alkyl)₃, —O—C₁₋₄₀ alkyl, a C₁₋₄₀ alkyl group, a C₂₋₄₀ alkenyl group, a C₂₋₄₀ alkynyl group, and a C₁₋₄₀ haloalkyl group; wherein each of the C₁₋₄₀ alkyl group, the C₂₋₄₀ alkenyl group, the C₂₋₄₀ alkynyl group, and the C₁₋₄₀ haloalkyl group optionally can be substituted with 1-5 substituents independently selected from a halogen, —CN, NO₂, OH, —NH₂, —NH(C₁₋₆ alkyl), —N(C₁₋₆ alkyl)₂, —S(O)₂OH, —CHO, —C(O)—C₁₋₆ alkyl, —C(O)OH, —C(O)—OC₁₋₆ alkyl, —C(O)NH₂, —C(O)NH—C₁₋₆ alkyl, —C(O)N(C₁₋₆ alkyl)₂, —OC₁₋₆ alkyl, —SiH₃, —SiH(C₁₋₆ alkyl)₂, —SiH₂(C₁₋₆ alkyl), and —Si(C₁₋₆ alkyl)₃.

To further illustrate, in certain embodiments, R¹ and R² independently can be selected from H or -L-R^{a}, where R^{a} is selected from a C₁₋₄₀ alkyl group, a C₂₋₄₀ alkenyl group, and a C₁₋₄₀ haloalkyl group, each of which optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃; and L is a covalent bond or a linker comprising one or more heteroatoms. For example, L can be a linker selected from —Y—O—Y—, —Y—[S(O)_{w}]—Y—, —Y—C(O)—Y—, —Y—[NR^{c}C(O)]—Y—, —Y—[C(O)NR^{C}]—, —Y—NR^{c}—Y—, —Y—[SiR^{c}₂]—Y—, where Y, at each occurrence, independently is selected from a divalent C₁₋₄₀ alkyl group, a divalent C₂₋₄₀ alkenyl group, a divalent C₁₋₄₀ haloalkyl group, and a covalent bond; R^{c} is selected from H, a C₁₋₆ alkyl group, a C₆₋₁₄ aryl group, and a —C₁₋₆ alkyl-C₆₋₁₄ aryl group; and w is 0, 1, or 2. In some embodiments, R¹ and R² independently can be selected from H, a C₃₋₄₀ alkyl group, a C₄₋₄₀ alkenyl group, and a C₃₋₄₀ haloalkyl group, and an —O—C₃₋₄₀ alkyl group, where each of these groups can be linear or branched, and can be optionally substituted as described herein.

In other embodiments, R¹ and R² independently can be an organic group that includes one or more cyclic moieties. For example, R¹ and R² independently can be selected from -L′-Cy¹, -L′-Cy¹-L′-Cy², -L′-Cy¹-L′-Cy²-Cy², -L′-Cy¹-Cy¹, -L′Cy¹-Cy¹-L′-Cy², -L′-Cy¹-Cy¹-L′-Cy²-Cy², -L′-Cy¹-L′-R^{a}, -L′-Cy¹-L′-Cy²-L-R^{a}, -L′-Cy¹-L′-Cy²-Cy²-L-R^{a}, -L′-Cy¹-Cy¹-L-R^{a}, and -L′-Cy¹-Cy¹-L′-Cy²-L-R^{a};

wherein:
Cy¹ and Cy² independently are selected from a C₆₋₁₄ aryl group, a 5-14 membered heteroaryl group, a C₃₋₁₄ cycloalkyl group, and a 3-14 membered cycloheteroalkyl group, each of which optionally can be substituted with 1-5 substituents independently selected from a halogen, —CN, oxo, ═C(CN)₂, a C₁₋₆ alkyl group, a C₁₋₆ alkoxy group, and a C₁₋₆ haloalkyl group;
L′, at each occurrence, independently is a covalent bond or a linker selected from —Y—O—Y—, —Y—[S(O)_{w}]—Y—, —Y—C(O)—Y—, —Y—[NR^{c}C(O)]—Y—, —Y—[C(O)NR^{c}]—, —Y—NR^{c}—Y—, —Y—[SiR^{c}₂]—Y—, a divalent C₁₋₄₀ alkyl group, a divalent C₂₋₄₀ alkenyl group, and a divalent C₁₋₄₀ haloalkyl group, where Y, R^{c}, and w are as defined above;
R^{a} is selected from a C₁₋₄₀ alkyl group, a C₂₋₄₀ alkenyl group, a C₂₋₄₀ alkynyl group, and a C₁₋₄₀ haloalkyl group, each of which optionally can be substituted with 1-10 substituents independently selected from a halogen, —CN, NO₂, OH, —NH₂, —NH(C₁₋₄₀ alkyl), —N(C₁₋₄₀alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃.

Further examples of R¹ and R² include:

1) linear or branched C₁₋₄₀ alkyl groups and C₂₋₄₀ alkenyl groups such as:

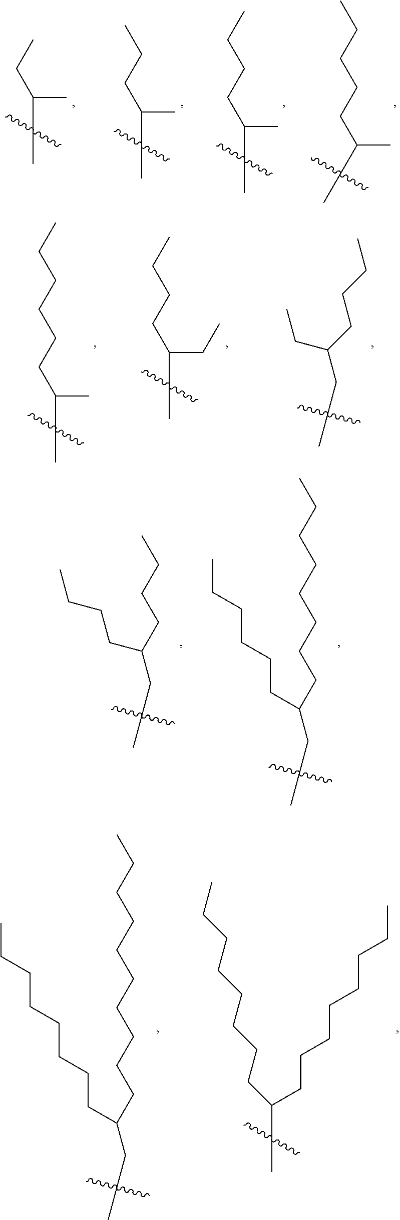

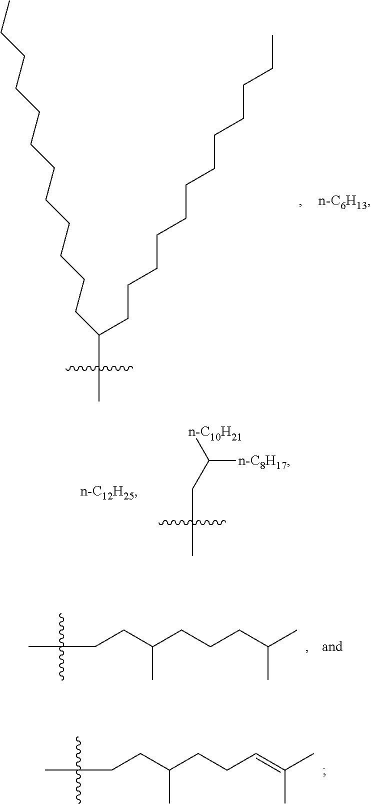

2) optionally substituted cycloalkyl groups such as:

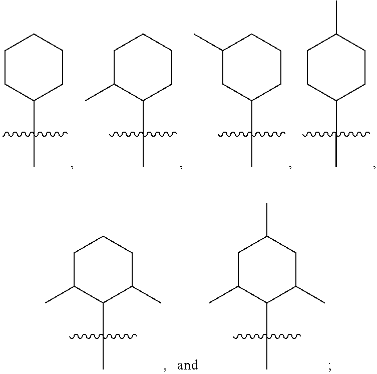

and
3) optionally substituted aryl groups, arylalkyl groups, biaryl groups, biarylalkyl groups such as:

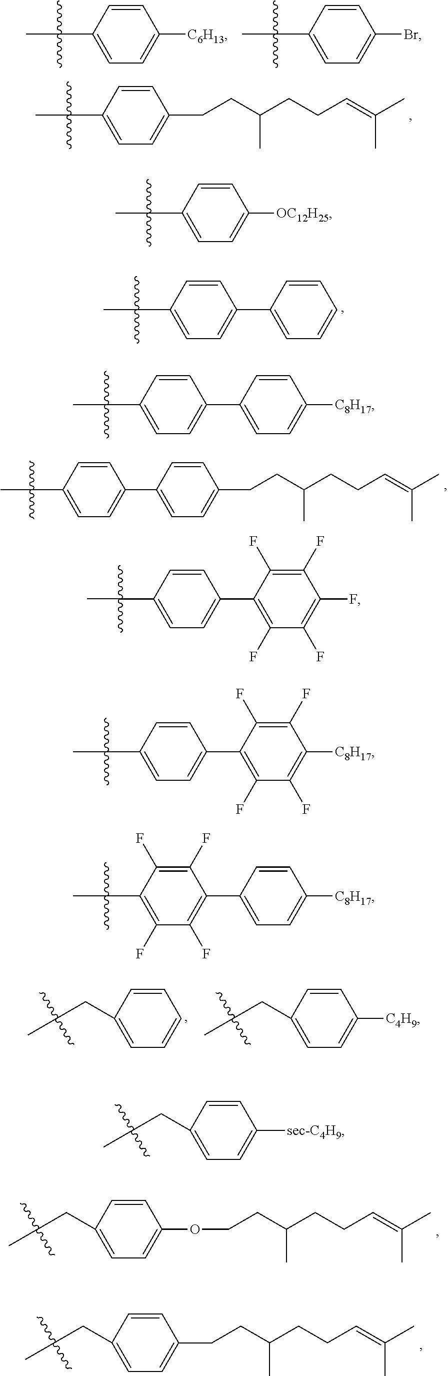

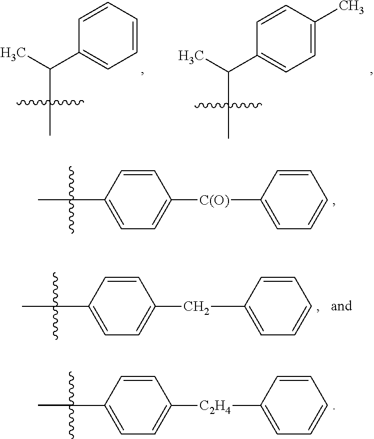

In various embodiments, R³ can be selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 membered heteroaryl group, the C₃₋₁₄ cycloalkyl group, and the 3-14 membered cycloheteroalkyl group optionally can be substituted with 1-10 substituents independently selected from a halogen, —CN, a C₁₋₁₀ alkyl group, a C₁₋₁₀ alkoxy group, and a C₁₋₁₀ haloalkyl group.

For example, R³ can be selected from Si(CH₃)₃, Si(OCH₃)₃, a cyclohexyl group, a linear or branched C₁₋₁₀ alkyl group (e.g., an n-octyl group), a linear or branched C₁₋₁₀ haloalkyl group, and a phenyl group optionally substituted with 1-5 substituents independently selected from a halogen, a C₁₋₆ alkyl group (e.g., a t-butyl group), a C₁₋₆ alkoxy group, and a C₁₋₆ haloalkyl group.

In one aspect, the present teachings relate to compounds of formula II:

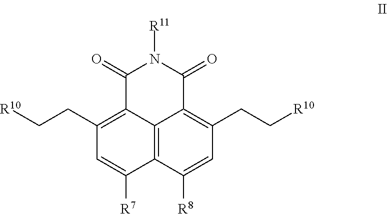

wherein:
R⁷ and R⁸ independently are selected from H, halogen, —CN, a C₁₋₁₀ alkyl group, a C₁₋₁₀ alkoxy group, and a C₁₋₁₀ haloalkyl group; or alternatively,
R⁷ and R⁸ together can be:

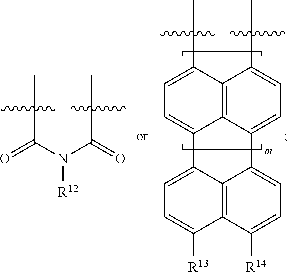

R¹⁰ is selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 membered heteroaryl group, the C₃₋₁₄ cycloalkyl group, and the 3-14 membered cycloheteroalkyl group optionally can be substituted with 1-10 substitutents independently selected from a halogen, —CN, a C₁₋₁₀ alkyl group, a C₁₋₁₀ alkoxy group, and a C₁₋₁₀ haloalkyl group;
R¹¹ and R¹² independently are selected from H, a C₁₋₄₀ alkyl group, a C₂₋₄₀ alkenyl group, a C₁₋₄₀ haloalkyl group, and an organic group comprising 1-4 cyclic moieties,
wherein:
each of the C₁₋₄₀ alkyl group, the C₂₋₄₀ alkenyl group, and the C₁₋₄₀ haloalkyl group optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group can be bonded covalently to the imide nitrogen atom via an optical linker; and
each of the 1-4 cyclic moieties in the organic group can be the same or different, can be bonded covalently to each other or the imide nitrogen via an optical linker, and optionally can be substituted with 1-5 substituents independently selected from a halogen, oxo, —CN, NO₂, OH, ═C(O)OH, —C(O)—C₁₋₄₀ alkyl, —C(O)—OC₁₋₄₀ alkyl, —C(O)NH₂, —C(O)NH—C₁₋₄₀ alkyl, —C(O)N(C₁₋₄₀ alkyl)₂, —SiH₃, —SiH₃(C₁₋₄₀ alkyl)₂, —SiH₂(C₁₋₄₀ alkyl), —Si(C₁₋₄₀ alkyl)₃, —O—C₁₋₄₀ alkyl, —O—C₁₋₄₀ alkenyl, —O—C₁₋₄₀ haloalkyl, a C₁₋₄₀ alkyl group, a C₁₋₄₀ alkenyl group, and a C₁₋₄₀ haloalkyl group;
   R¹³ and R¹⁴ independently are selected from H, halogen, —CN, a C₁₋₄₀ alkyl group, a C₁₋₁₀ alkoxy group, and a C₁₋₁₀ haloalkyl group; and
   m is 0, 1, 2, or 3.

In certain embodiments, R⁷ and R⁸ independently can be selected from H, halogen, —CN, a C₁₋₁₀ alkyl group, a C₁₋₁₀ alkoxy group, and a C₁₋₁₀ haloalkyl group. Compounds of formula II according to these embodiments can be referred as naphthalene-1,8-dicarboximides (or simply, naphthalene imides) that are substituted with two —(CH₂)₂R¹⁰ groups at positions ortho to the imide groups, where R¹⁰ and R¹¹ are as defined herein.

In certain embodiments, R⁷ and R⁸ together can be:

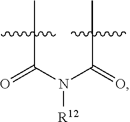

where R¹² is as defined herein. Compounds of formula II according to these embodiments can be represented by:

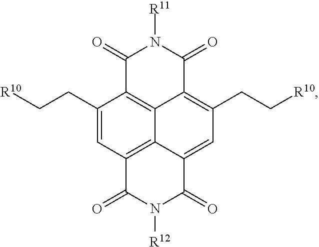

that is, naphthalene-1,8:4,5-bis(dicarboximides) (or simply, naphthalene diimides) that are substituted with two —(CH₂)₂R¹⁰ groups at positions ortho to the imide groups, where R¹⁰, R¹¹, and R¹² are as defined herein.

In certain embodiments, R⁷ and R⁸ together can be:

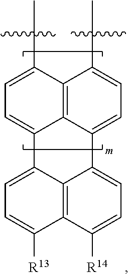

where R¹³ and R¹⁴ are as defined herein. Compounds of formula II according to these embodiments can be represented by:

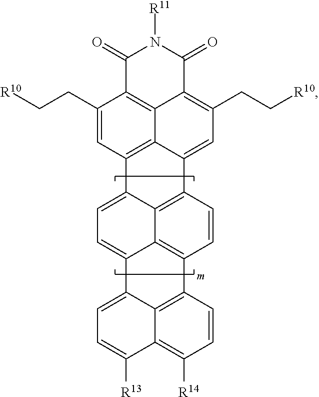

where R¹⁰, R¹¹, R¹³, R¹⁴, and m are as defined herein.

In particular embodiments, m can be 0. Compounds according to such embodiments can be represented by:

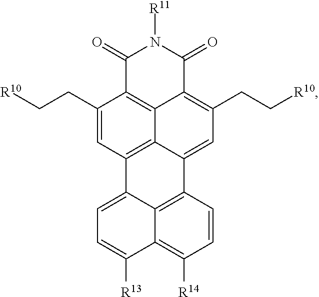

that is, perylene-3,4-dicarboximides (or simply, perylene imides) that are substituted with two —(CH₂)₂R¹⁰ groups at positions ortho to the imide groups, where R¹⁰, R¹¹, R¹³, and R¹⁴ are as defined herein.

In other embodiments, m can be 1. Compounds according to such embodiments can be represented by:

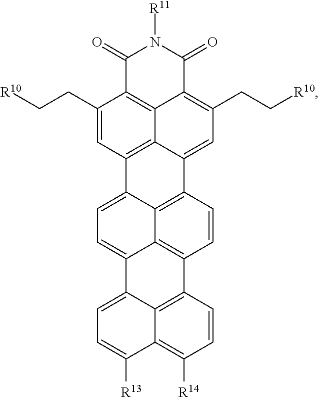

that is, terrylene-3,4-dicarboximides (or simply, terrylene imides) that are substituted with two —(CH₂)₂R¹⁰ groups at positions ortho to the imide groups, where R¹⁰, R¹¹, R¹³, and R¹⁴ are as defined herein.

In various embodiments, R¹¹ (and R¹² if present) can be H or a substituent as described herein. For example, in various embodiments, R¹¹ and R¹² independently can be H or a substituent which can impart improved desirable properties to the compound as a whole. For example, certain substituents including one or more electron-withdrawing or electron-donating moieties can modulate the electronic properties of the compound, while substituents that include one or more aliphatic chains can improve the solubility of the compound in organic solvents.

Accordingly, in certain embodiments, R¹¹ and R¹² independently can be a linear or branched C₃₋₄₀ alkyl group, examples of which include an n-hexyl group, an n-octyl group, an n-dodecyl group, a 1-methylpropyl group, a 1-methylbutyl group, a 1-methylpentyl group, a 1-methylhexyl group, a 1-ethylpropyl group, a 1-ethylbutyl group, a 1,3-dimethylbutyl group, a 2-ethylhexyl group, a 2-hexyloctyl group, a 2-octyldodecyl group, and a 2-decyltetradecyl group. In certain embodiments, R¹¹ and R¹² independently can be a linear or branched C₃₋₄₀ alkenyl group (such as the linear or branched C₃₋₄₀ alkyl groups specified above but with one or more saturated bonds replaced by unsaturated bonds). In particular embodiments, R¹¹ and R¹² independently can be a branched C₃₋₂₀ alkyl group or a branched C₃₋₂₀ alkenyl group.

In certain embodiments, R¹¹ and R¹² independently can be a linear or branched C₆₋₄₀ alkyl or alkenyl group, an arylalkyl group (e.g., a benzyl group) substituted with a linear or branched C₆₋₄₀ alkyl or alkenyl group, an aryl group (e.g., a phenyl group) substituted with a linear or branched C₆₋₄₀ alkyl or alkenyl group, or a biaryl group (e.g., a biphenyl group) substituted with a linear or branched C₆₋₄₀ alkyl or alkenyl group, wherein each of these groups optionally can be substituted with 1-5 halo groups (e.g., F). In some embodiments, R¹¹ and R¹² independently can be a biaryl group wherein the two aryl groups are covalently linked via a linker. For example, the linker can be a divalent C₁₋₄₀ alkyl group wherein one or more non-adjacent CH₂ groups optionally can be replaced by —O—, —S—, or —Se—, i.e., O, S, and/or Se atoms are not linked directly to one another. The linker can include other heteroatoms and/or functional groups as described herein.

More generally, R¹¹ and R¹² independently can be selected from H, a C₁₋₄₀ alkyl group, a C₂₋₄₀ alkenyl group, a C₁₋₄₀ haloalkyl group, and an organic group comprising 1-4 cyclic moieties, wherein
each of the C₁₋₄₀ alkyl group, the C₂₋₄₀ alkenyl group, and the C₁₋₄₀ haloalkyl group optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group can be covalently bonded to the imide nitrogen atom via an optical linker; and
each of the 1-4 cyclic moieties in the organic group can be the same or different, can be bonded covalently to each other or the imide nitrogen via an optional linker, and optionally can be substituted with 1-5 substituents independently selected from a halogen, —CN, oxo, NO₂, OH, ═C(CN)₂, —NH₂, —NH(C₁₋₄₀ alkyl), —N(C₁₋₄₀ alkyl)₂, —S(O)₂OH, —CHO, —C(O)OH, —C(O)—C₁₋₄₀ alkyl, —C(O)—OC₁₋₄₀ alkyl, —C(O)NH₂, —C(O)NH—C₁₋₄₀ alkyl, —C(O)N(C₁₋₄₀ alkyl)₂, —SiH₃, —SiH(C₁₋₄₀ alkyl)₂, —SiH₂(C₁₋₄₀ alkyl), —Si(C₁₋₄₀ alkyl)₃, —O—C₁₋₄₀ alkyl, a C₁₋₄₀ alkyl group, a C₂₋₄₀ alkenyl group, a C₂₋₄₀ alkynyl group, and a C₁₋₄₀ haloalkyl group; wherein each of the C₁₋₄₀ alkyl group, the C₂₋₄₀ alkenyl group, the C₂₋₄₀ alkynyl group, and the C₁₋₄₀ haloalkyl group optionally can be substituted with 1-5 substituents independently selected from a halogen, —CN, NO₂, OH, —NH₂, —NH(C₁₋₆ alkyl), —N(C₁₋₆ alkyl)₂, —S(O)₂OH, —CHO, —C(O)—C₁₋₆ alkyl, —C(O)OH, —C(O)—OC₁₋₆ alkyl, —C(O)NH₂, —C(O)NH—C₁₋₆ alkyl, —C(O)N(C₁₋₆ alkyl)₂, —OC₁₋₆ alkyl, —SiH₃, —SiH(C₁₋₆ alkyl)₂, —SiH₂(C₁₋₆ alkyl), and —Si(C₁₋₆ alkyl)₃.

To further illustrate, in certain embodiments, R¹¹ and R¹² independently can be selected from H or -L-R^{a}, where R^{a} is selected from a C₁₋₄₀ alkyl group, a C₂₋₄₀ alkenyl group, and a C₁₋₄₀ haloalkyl group, each of which optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃; and L is a covalent bond or a linker comprising one or more heteroatoms. For example, L can be a linker selected from —Y—O—Y—, —Y—[S(O)_{w}]—Y—, —Y—C(O)—Y—, —Y—[NR^{c}C(O)]—Y—, —Y—[C(O)NR^{c}]—, —Y—NR^{c}—Y—, —Y—[SiR^{c}₂]—Y—, where Y, at each occurrence, independently is selected from a divalent C₁₋₄₀ alkyl group, a divalent C₂₋₄₀ alkenyl group, a divalent C₁₋₄₀ haloalkyl group, and a covalent bond; R^{c} is selected from H, a C₁₋₆ alkyl group, a C₆₋₁₄ aryl group, and a —C₁₋₆ alkyl-C₆₋₁₄ aryl group; and w is 0, 1, or 2. In some embodiments, R¹¹ and R¹² independently can be selected from H, a C₃₋₄₀ alkyl group, a C₄₋₄₀ alkenyl group, a C₄₋₄₀ alkynyl group, and a C₃₋₄₀ haloalkyl group, and an —O—C₃₋₄₀ alkyl group, where each of these groups can be linear or branched, and can be optionally substituted as described herein.

In other embodiments, R¹¹ and R¹² independently can be an organic group that includes one or more cyclic moieties. For example, R¹¹ and R¹² independently can be selected from -L′-Cy¹, -L′-Cy¹-L′-Cy², -L′-Cy¹-L′-Cy²-Cy², -L′-Cy¹-Cy¹, -L′-Cy¹-Cy¹-L′-Cy², -L′-Cy¹-Cy¹-L′-Cy²-Cy², -L′-Cy¹-L-R^{a}, -L′-Cy¹-L′-Cy²-L-R^{a}, -L′-Cy¹-L′-Cy²-Cy²-L-R^{a}, -L′-Cy¹-Cy¹-L-R^{a}, and -L′-Cy¹-Cy¹-L′-Cy²-L-R^{a}; wherein:

Cy¹ and Cy² independently are selected from a C₆₋₁₄ aryl group, a 5-14 membered heteroaryl group, a C₃₋₁₄ cycloalkyl group, and a 3-14 membered cycloheteroalkyl group, each of which optionally can be substituted with 1-5 substituents independently selected from a halogen, —CN, oxo, ═C(CN)₂, a C₁₋₆ alkyl group, a C₁₋₆ alkoxy group, and a C₁₋₆ haloalkyl group;
L′, at each occurrence, independently is a covalent bond or a linker selected from —Y—O—Y—, —Y—[S(O)_{w}]—Y—, —Y—C(O)—Y—, —Y—[NR^{c}C(O)]—Y—, —Y—[C(O)NR^{c}]—, —Y—NR^{c}—Y—, —Y—[SiR^{c}₂]—Y—, a divalent C₁₋₄₀ alkyl group, a divalent C₂₋₄₀ alkenyl group, and a divalent C₁₋₄₀ haloalkyl group, where Y, R^{c}, and w are as defined above;
R^{a} is selected from a C₁₋₄₀ alkyl group, a C₂₋₄₀ alkenyl group, a C₂₋₄₀ alkynyl group, and a C₁₋₄₀ haloalkyl group, each of which optionally can be substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃.

Further examples of R¹¹ and R¹² include:

1) linear or branched C₁₋₄₀ alkyl groups and C₂₋₄₀ alkenyl groups such as:

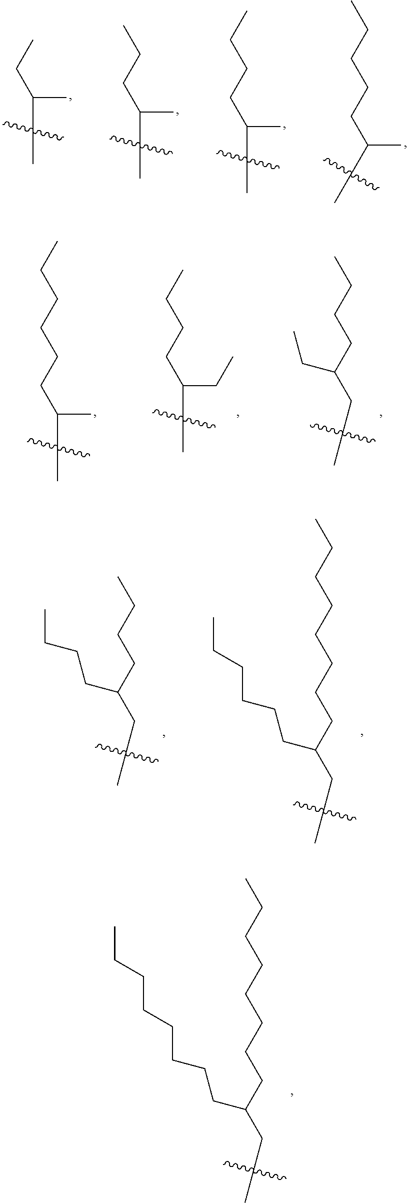

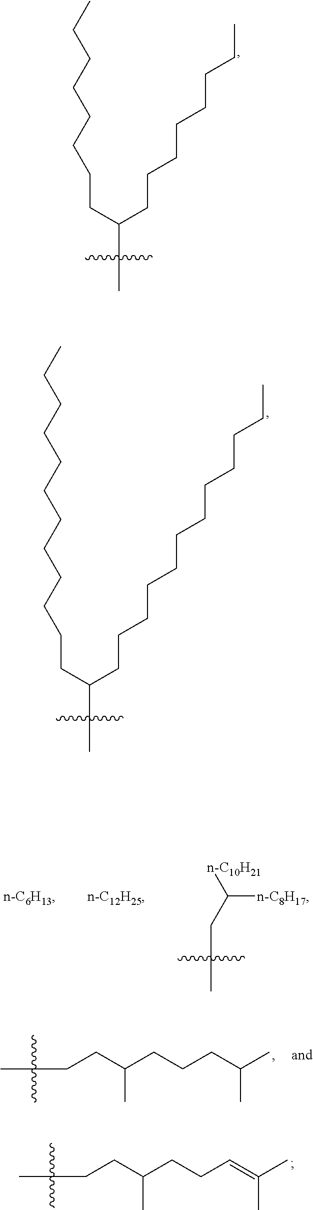

2) optionally substituted cycloalkyl groups such as:

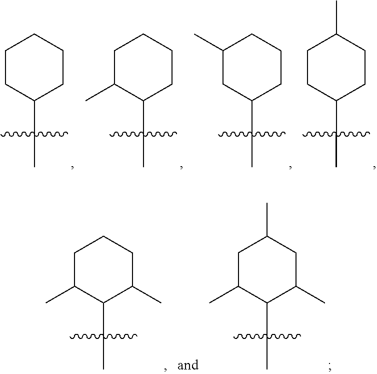

and
3) optionally substituted aryl groups, arylalkyl groups, biaryl groups, biarylalkyl groups such as:

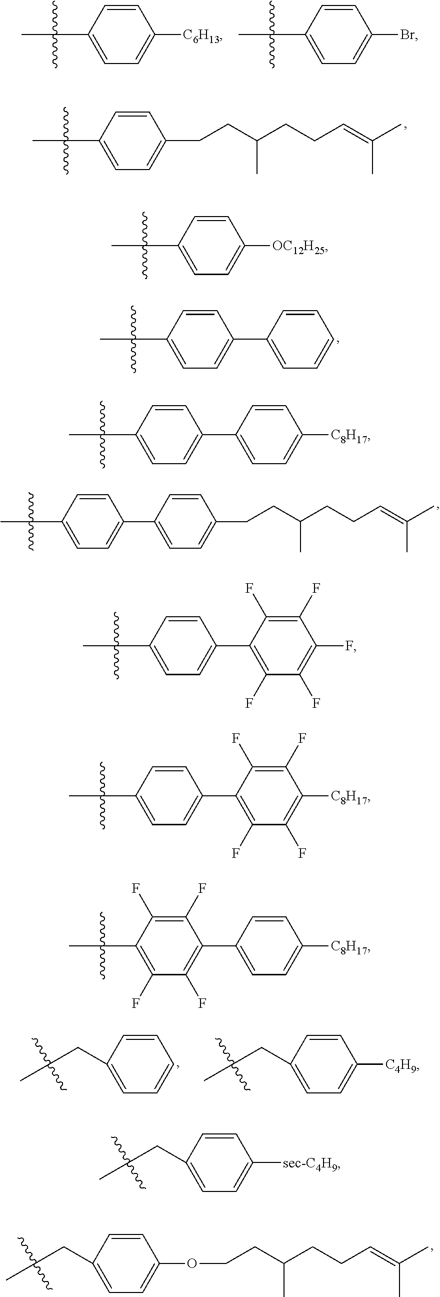

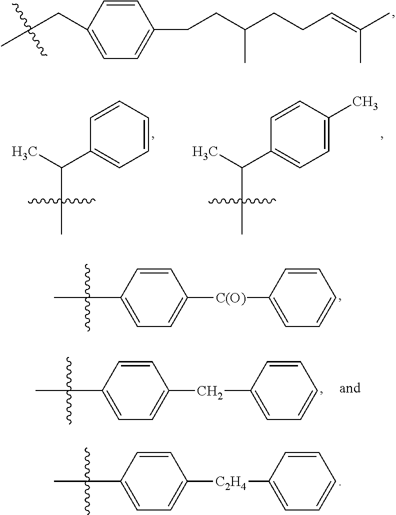

In various embodiments, R¹⁰ can be selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 membered heteroaryl group, the C₃₋₁₄ cycloalkyl group, and the 3-14 membered cycloheteroalkyl group optionally can be substituted with 1-10 substituents independently selected from a halogen, —CN, a C₁₋₁₀ alkyl group, a C₁₋₁₀ alkoxy group, and a C₁₋₁₀ haloalkyl group.

For example, R¹⁰ can be selected from Si(CH₃)₃, Si(OCH₃)₃, a cyclohexyl group, a linear or branched C₁₋₁₀ alkyl group (e.g., an n-octyl group), a linear or branched C₁₋₁₀ haloalkyl group, and a phenyl group optionally substituted with 1-5 substituents independently selected from a halogen, a C₁₋₆ alkyl group (e.g., a t-butyl group), a C₁₋₆ alkoxy group, and a C₁₋₆ haloalkyl group.

For naphthalene imides and perylene imides according to the present teachings, R⁷, R⁸, R¹³, and R¹⁴ independently can be selected from H, halogen, —CN, a C₁₋₁₀ alkyl group, a C₁₋₁₀ alkoxy group, and a C₁₋₁₀ haloalkyl group. In various embodiments, each of R⁷ and R⁸ and each of R¹³ and R¹⁴ can be H.

By way of example, compounds according to the present teachings can be prepared in accordance with the procedures outlined in Schemes 1 and 2 below.

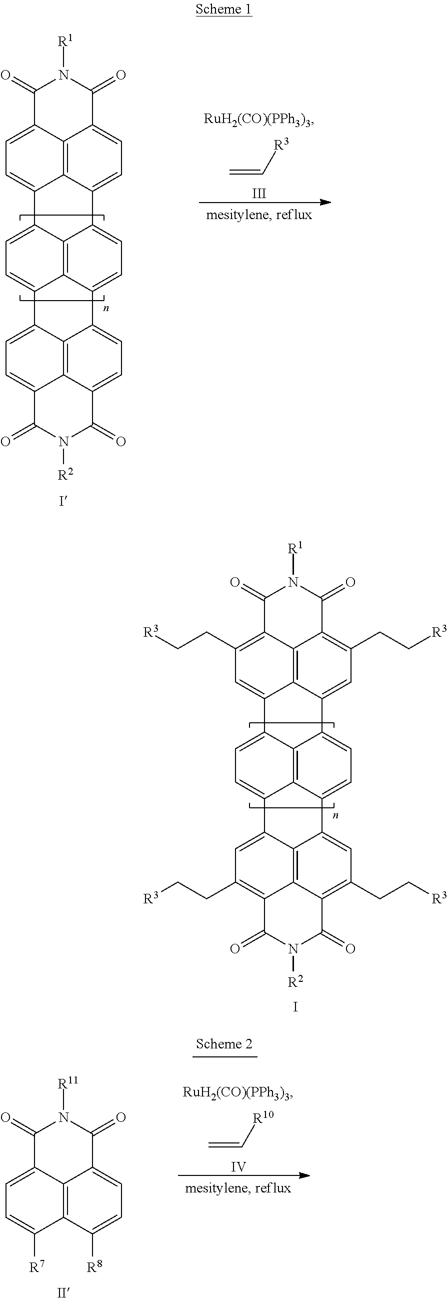

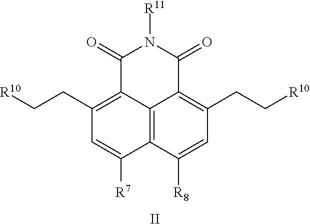

Referring to Schemes 1 and 2, compounds of formula I (or II) can be prepared by reacting a compound of formula I′ (or II′) with a vinyl compound of formula III (or IV) in the presence of a ruthenium (Ru) catalyst, where R¹, R², R³, R⁷, R⁸, R¹⁰, R¹¹, and n are as defined herein. A compound of formula I′ (or II′) can be prepared by reacting the corresponding anhydride with an appropriate amine, e.g., NH₂R¹ (or NH₂R² or NH₂R¹¹) where R¹, R², and R¹¹ are as defined herein. In various embodiments, the ruthenium catalyst can be RuH₂(CO)(PPh₃)₃. The reaction can be performed in aromatic hydrocarbons such as benzene, toluene, xylene, and mesitylene, or any other solvent system in which the compounds have sufficient solubility. Compounds of formula III (or IV) can be provided in excess, e.g., at least five times the molar amount of compound I′ (or II′) based on the expected number of alkylation. The reaction typically is performed at an elevated temperature, e.g., around the boiling point of the solvent(s).

Referring to Scheme 2 above, where compounds of formula II and II′ are naphthalene diimides, it was found that four-fold alkylation does not occur as expected. Without wishing to be bound by any particular theory, it is believed that steric hindrance prevents the Ru catalyst from effectively binding to the carbonyl group to activate the C—H bond to facilitate coupling to the vinyl compound in the presence of an adjacent substitutent, which inhibits the vinyl compound from coordinating with the Ru to form the C—C bond. Instead, a 1:1 isomeric mixture of 2,6- and 2,7-disubstituted products is obtained as illustrated in Scheme 3 below.

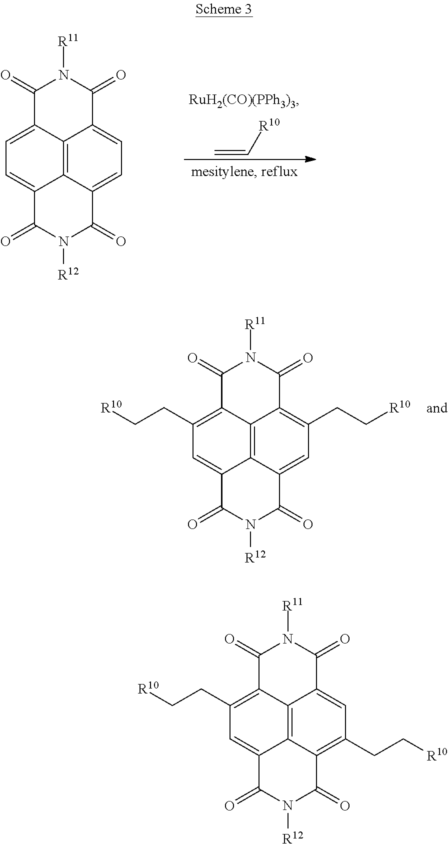

Table 1 provides exemplary compounds according to the present teachings and exemplary reaction time and yield when the compounds were prepared according to the synthetic schemes described above.

| | Time | Yield | | |
|---|---|---|---|---|
| Reagent | (hr) | (%) | Compound | |
| N-(n-octyl)- naphthalene-1,8- dicarboximide (1) | 24 | 49 | N-(n-octyl)-2,7- bis(phenylethyl)- naphthalene-1,8- dicarboximide (2) | 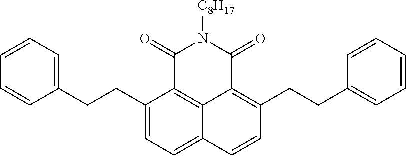 |
| N,N′-bis(n-octyl)- naphthalene- 1,8:4,5- bis(dicarboximide) (3) | 2.5 | 24 | N,N′-bis(n-octyl)- 2,7-bis(phenylethyl)- naphthalene-1,8:4,5- bis(dicarboximide) (4) | 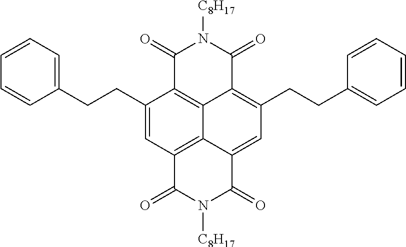 |
| N-(2′-ethylhexyl)- perylene-3,4- dicarboximide (5) | 48 | 72 | N-(2′- ethylhexyl)- bis(2,7-phenylethyl)- perylene-3,4- dicarboximide (6) | 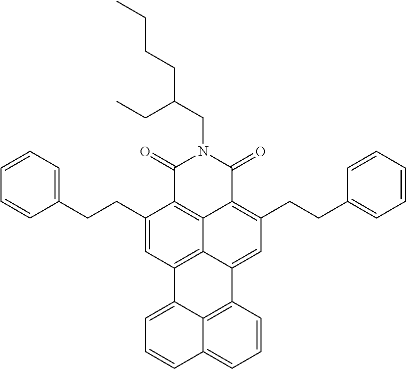 |
| N,N′-bis(n-octyl)- perylene-3,4:9,10- bis(dicarboximide) (7) | 72 | 75 | N,N′-bis(n-octyl)- 2,5,8,11- tetrakis(phenylethyl)- perylene-3,4:9,10- bis(dicarboximide) (8) | 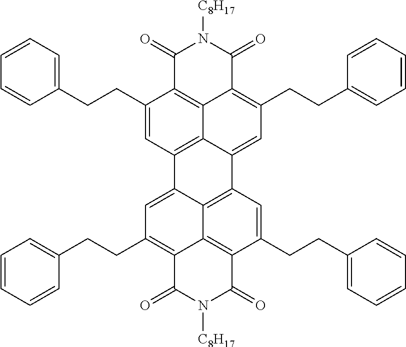 |
| N,N′-bis(2- ethylhexyl)- terrylene- 3,4:11,12- bis(dicarboximide) (9) | 48 | 81 | N,N′-bis(2- ethylhexyl)- 2,5,10,13- tetrakis(phenylethyl)- terrylene-3,4:11,12- bis(dicarboximide) (10) | 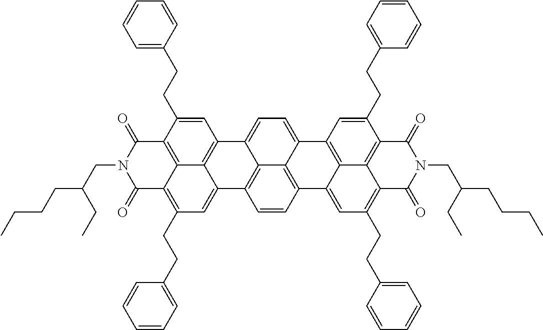 |
| N-(2′-ethylhexyl)- perylene-3,4- dicarboximide (11) | 120 | 82 | N-(2′-ethylhexyl)- 2,7-didodecyl- perylene-3,4- dicarboximide (12) | 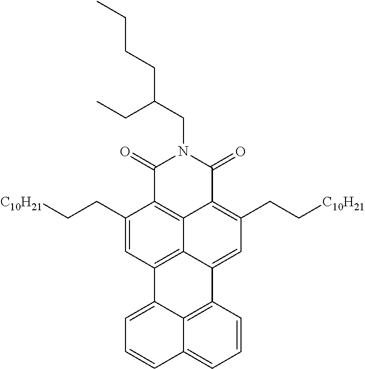 |
| N-(2′-ethylhexyl)- perylene-3,4- dicarboximide (13) | 48 | 99 | N-(2′-ethylhexyl)- 2,5-bis(4-tert-butyl- phenyl-ethyl)- perylene-3,4- dicarboximide (14) | 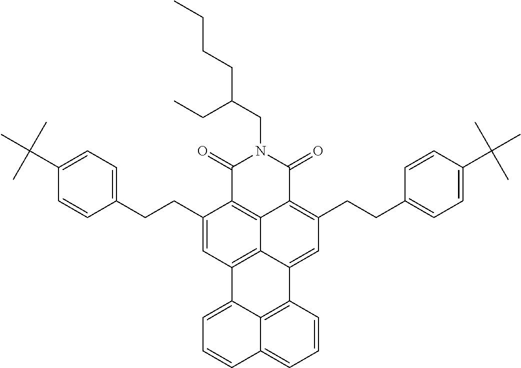 |
| N,N′-bis(2- ethylhexyl)- perylene-3,4:9,10- bis(dicarboximide) (15) | 48 | 72 | N,N′-bis(2- ethylhexyl)-2,5,8,11- tetrakis(4-tert-butyl- phenyl-ethyl)- perylene-3,4:9,10- bis(dicarboximide) (16) | 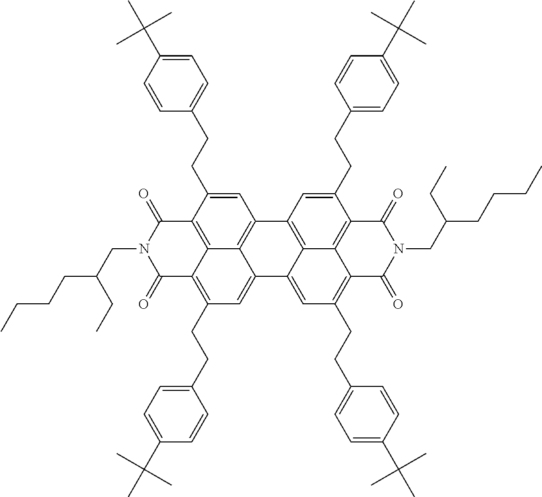 |
| N-(2-ethylhexyl)- N′(4- bromophenyl)- perylene- 3,4:11,12- bis(dicarboximide) (17) | 168 | 83 | N-(2-ethylhexyl)- N′(4-bromophenyl)- 2,5,8,11- tetradodecyl- perylene-3,4:11,12- bis(dicarboximide) (18) | 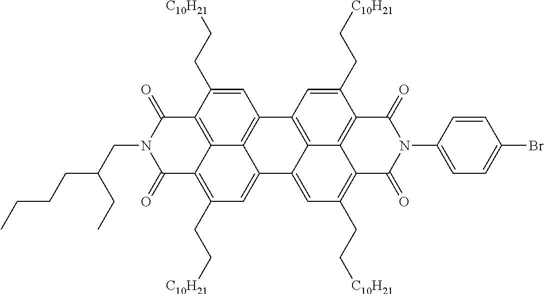 |
| N-(2-ethylhexyl)- N′(4- bromophenyl)- perylene- 3,4:11,12- bis(dicarboximide) (19) | 48 | 74 | N-(2-ethylhexyl)- N′(4-bromophenyl)- 2,5,8,11- tetrakis(phenylethyl)- perylene-3,4:11,12- bis(dicarboximide) (20) | 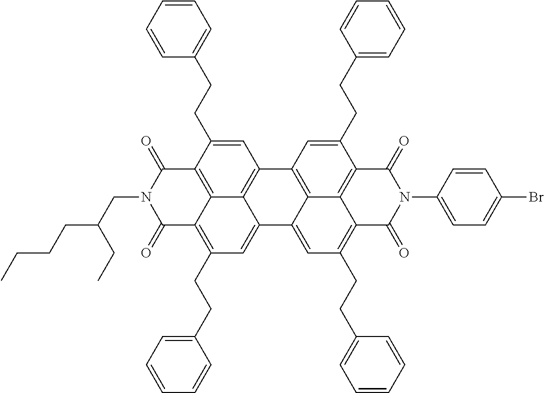 |
| N-(2-ethylhexyl)- N′(4- bromophenyl)- perylene- 3,4:11,12- bis(dicarboximide) (21) | 48 | 53 | N-(2-ethylhexyl)- N′(4-bromophenyl)- 2,5,8,11-tetrakis(4- tert-butyl-phenyl- ethyl)-perylene- 3,4:11,12- bis(dicarboximide) (22) | 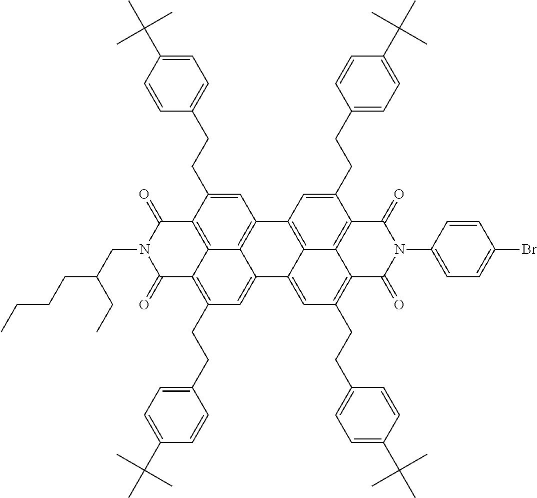 |
| N,N′-bis(2- ethylhexyl)- terrylene-3,4:11,12- bis(dicarboximide) (23) | 168 | 33 | N,N′-bis(2- ethylhexyl)- 2,5,10,13- tetradodecyl- terrylene-3,4:11,12- bis(dicarboximide) (24) | 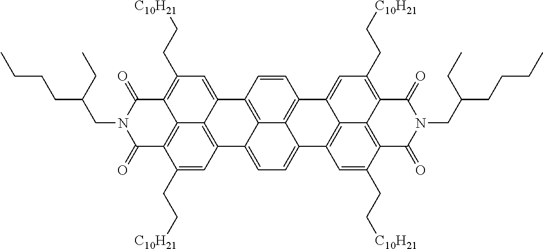 |
| N,N′-bis(2- ethylhexyl)- terrylene-3,4:11,12- bis(dicarboximide) (25) | 48 | 79 | N,N′-bis(2- ethylhexyl)- 2,5,10,13-tetrakis(4- tert-butyl-phenyl- ethyl)-terrylene- 3,4:11,12- bis(dicarboximide) (26) | 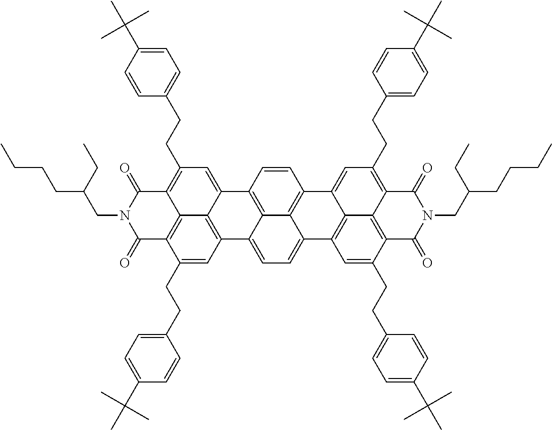 |

Alternatively, the present compounds can be prepared from commercially available starting materials, compounds known in the literature, or via other readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The present compounds can have significantly higher solubility compared to otherwise similar but unsubstituted (i.e., no substitution at positions ortho to the imide groups) rylene imide and diimides. As used herein, a compound can be considered soluble in a solvent when at least 0.1 mg of the compound can be dissolved in 1 mL of the solvent. Examples of common organic solvents include petroleum ethers; acetonitrile; aromatic hydrocarbons such as benzene, toluene, xylene, and mesitylene; ketones such as acetone, and methyl ethyl ketone; ethers such as tetrahydrofuran, dioxane, bis(2-methoxyethyl)ether, diethyl ether, di-isopropyl ether, and t-butyl methyl ether; alcohols such as methanol, ethanol, butanol, and isopropyl alcohol; aliphatic hydrocarbons such as hexanes; esters such as methyl acetate, ethyl acetate, methyl formate, ethyl formate, isopropyl acetate, and butyl acetate; amides such as dimethylformamide and dimethylacetamide; sulfoxides such as dimethylsulfoxide; halogenated aliphatic and aromatic hydrocarbons such as dichloromethane, chloroform, ethylene chloride, chlorobenzene, dichlorobenzene, and trichlorobenzene; and cyclic solvents such as cyclopentanone, cyclohexanone, and 2-methypyrrolidone. In various embodiments, the present compounds can have good solubility in solvents that are aromatic hydrocarbons such as benzene, toluene, xylene, and mesitylene.

For example, N-(2′-ethylhexyl)-2,5-bis(phenethyl)perylene-3,4-dicarboximide (6) is three times more soluble than N-(2′-ethylhexyl)-perylene-3,4-dicarboximide (5). Table 2 below compares the solubilities of several compounds according to the present teachings with their unsubstituted counterparts in toluene.

TABLE 2

| | Solubility in |
|---|---|
| Compound | toluene (mg/mL) |
| N-(2′-ethylhexyl)-perylene-3,4-dicarboximide (5) | 2 |
| N-(2′-ethylhexyl)-bis(2,7-phenylethyl)- | 6 |
| perylene-3,4-dicarboximide (6) | |
| N,N′-bis(n-octyl)-perylene-3,4: 9,10- | <1 |
| bis(dicarboximide) (7) | |
| N,N′-bis(n-octyl)-2,5,8,11- | 2 |
| tetrakis(phenylethyl)-perylene-3,4: 9,10- | |
| bis(dicarboximide) (8) | |
| N,N′-bis(2-ethylhexyl)-terrylene-3,4: 11,12- | <1 |
| bis(dicarboximide) (9) | |
| N,N′-bis(2-ethylhexyl)-2,5,10,13- | 3 |
| tetrakis(phenylethyl)-terrylene-3,4: 11,12- | |
| bis(dicarboximide) (10) | |

The processes described herein can be monitored according to any suitable method known in the art. For example, product formation can be monitored by spectroscopic means, such as nuclear magnetic resonance spectroscopy (NMR, e.g., ¹H or ¹³C), infrared spectroscopy (IR), spectrophotometry (e.g., UV-visible), mass spectrometry (MS), or by chromatography such as high pressure liquid chromatography (HPLC), gas chromatography (GC), gel-permeation chromatography (GPC), or thin layer chromatography (TLC).

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Certain embodiments disclosed herein can be stable in ambient conditions (“ambient stable”) and soluble in common solvents. As used herein, a compound can be considered electrically “ambient stable” or “stable at ambient conditions” when a transistor (e.g., organic thin film transistor, OTFT) incorporating the compound as its semiconducting material exhibits a carrier mobility that is maintained at about its initial measurement when the compound is exposed to ambient conditions, for example, air, ambient temperature, and humidity, over a period of time. For example, a compound according to the present teachings can be described as ambient stable if a transistor incorporating the compound shows a carrier mobility that does not vary more than 20% or more than 10% from its initial value after exposure to ambient conditions, including, air, humidity and temperature, over a 3 day, 5 day, or 10 day period. In addition, a compound can be considered ambient stable if the optical absorption of the corresponding film does not vary more than 20% (preferably, does not vary more than 10%) from its initial value after exposure to ambient conditions, including air, humidity and temperature, over a 3 day, 5 day, or 10 day period.

OTFTs based on the present compounds can have long-term operability and continued high-performance in ambient conditions. For example, OTFTs based on certain embodiments of the present compounds can maintain satisfactory device performance in highly humid environment. Certain embodiments of the present compounds also can exhibit excellent thermal stability over a wide range of annealing temperatures. Photovoltaic devices can maintain satisfactory power conversion efficiencies over an extended period of time.

The present compounds can be fabricated into various articles of manufacture using solution processing techniques in addition to other more expensive processes such as vapor deposition. Various solution processing techniques have been used with organic electronics. Common solution processing techniques include, for example, spin coating, drop-casting, zone casting, dip coating, blade coating, or spraying. Another example of solution processing technique is printing. As used herein, “printing” includes a noncontact process such as inkjet printing, microdispensing and the like, and a contact process such as screen-printing, gravure printing, offset printing, flexographic printing, lithographic printing, pad printing, microcontact printing and the like.

Compounds of the present teachings can be used alone or in combination with other compounds to prepare semiconductor materials (e.g., compositions and composites), which in turn can be used to fabricate various articles of manufacture, structures, and devices. In some embodiments, semiconductor materials incorporating one or more compounds of the present teachings can exhibit n-type semiconductor activity, p-type semiconductor activity, ambipolar activity, light absorption, and/or light emission.

The present teachings, therefore, further provide methods of preparing a semiconductor material. The methods can include preparing a composition that includes one or more compounds disclosed herein dissolved or dispersed in a liquid medium such as a solvent or a mixture of solvents, depositing the composition on a substrate to provide a semiconductor material precursor, and processing (e.g., heating) the semiconductor precursor to provide a semiconductor material (e.g., in the form of a thin film or thin film semiconductor) that includes a polymer disclosed herein. In various embodiments, the liquid medium can be an organic solvent, an inorganic solvent such as water, or combinations thereof. In some embodiments, the composition can further include one or more additives independently selected from viscosity modulators, detergents, dispersants, binding agents, compatiblizing agents, curing agents, initiators, humectants, antifoaming agents, wetting agents, pH modifiers, biocides, and bactereriostats. For example, surfactants and/or polymers (e.g., polystyrene, polyethylene, poly-alpha-methylstyrene, polyisobutene, polypropylene, polymethylmethacrylate, and the like) can be included as a dispersant, a binding agent, a compatiblizing agent, and/or an antifoaming agent. In some embodiments, the depositing step can be carried out by printing, including inkjet printing and various contact printing techniques (e.g., screen-printing, gravure printing, offset printing, pad printing, lithographic printing, flexographic printing, and microcontact printing). In other embodiments, the depositing step can be carried out by spin coating, drop-casting, zone casting, dip coating, blade coating, or spraying.

Various articles of manufacture including electronic devices, optical devices, and optoelectronic devices, such as thin film semiconductors, field effect transistors (e.g., thin film transistors), photovoltaics, photodetectors, organic light emitting devices such as organic light emitting diodes (OLEDs) and organic light emitting transistors (OLETs), complementary metal oxide semiconductors (CMOSs), complementary inverters, diodes, capacitors, sensors, D flip-flops, rectifiers, and ring oscillators, that make use of the compounds disclosed herein are within the scope of the present teachings as are methods of making the same. The present compounds can offer processing and operation advantages in the fabrication and/or the use of these devices.

For example, articles of manufacture such as the various devices described herein can include a composite having a semiconductor material of the present teachings and a substrate component and/or a dielectric component. The substrate component can be selected from doped silicon, an indium tin oxide (ITO), ITO-coated glass, ITO-coated polyimide or other plastics, aluminum or other metals alone or coated on a polymer or other substrate, a doped polythiophene, and the like. The dielectric component can be prepared from inorganic dielectric materials such as various oxides (e.g., SiO₂, Al₂O₃, HfO₂), organic dielectric materials such as various polymeric materials (e.g., polycarbonate, polyester, polystyrene, polyhaloethylene, polyacrylate), and self-assembled superlattice/self-assembled nanodielectric (SAS/SAND) materials (e.g., as described in Yoon, M-H. et al., PNAS, 102 (13): 4678-4682 (2005), the entire disclosure of which is incorporated by reference herein), as well as hybrid organic/inorganic dielectric materials (e.g., described in U.S. patent application Ser. No. 11/642,504, the entire disclosure of which is incorporated by reference herein). In some embodiments, the dielectric component can include the crosslinked polymer blends described in U.S. patent application Ser. Nos. 11/315,076, 60/816,952, and 60/861,308, the entire disclosure of each of which is incorporated by reference herein. The composite also can include one or more electrical contacts. Suitable materials for the source, drain, and gate electrodes include metals (e.g., Au, Al, Ni, Cu), transparent conducting oxides (e.g., ITO, IZO, ZITO, GZO, GIO, GITO), and conducting polymers (e.g., poly(3,4-ethylenedioxythiophene) poly(styrenesulfonate) (PEDOT:PSS), polyaniline (PANI), polypyrrole (PPy)). One or more of the composites described herein can be embodied within various organic electronic, optical, and optoelectronic devices such as organic thin film transistors (OTFTs), specifically, organic field effect transistors (OFETs), as well as sensors, capacitors, unipolar circuits, complementary circuits (e.g., inverter circuits), and the like.

Accordingly, an aspect of the present teachings relates to methods of fabricating an organic field effect transistor that incorporates a semiconductor material of the present teachings. The semiconductor materials of the present teachings can be used to fabricate various types of organic field effect transistors including top-gate top-contact capacitor structures, top-gate bottom-contact capacitor structures, bottom-gate top-contact capacitor structures, and bottom-gate bottom-contact capacitor structures.

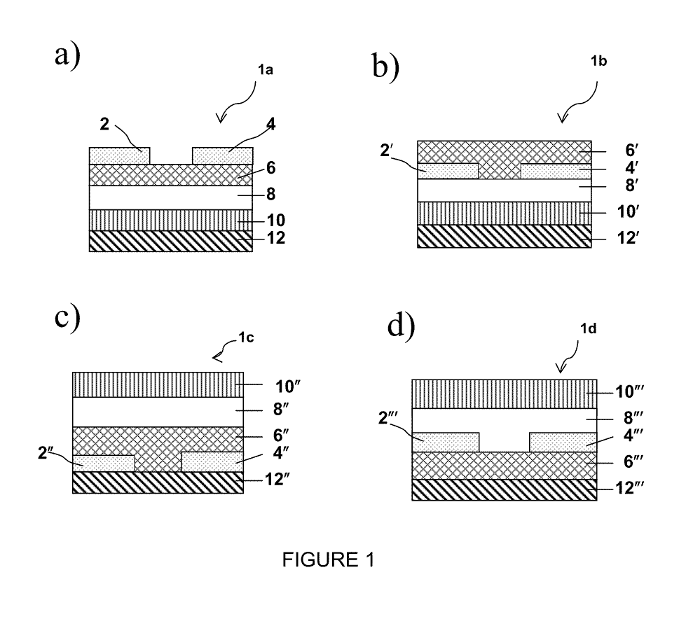

FIGS. 1a-d illustrates the four common types of OFET structures: (FIG. 1a) bottom-gate top-contact structure 1a, (FIG. 1b) bottom-gate bottom-contact structure 1b, (FIG. 1c) top-gate bottom-contact structure 1c, and (FIG. 1d) top-gate top-contact structure 1d. As shown in FIGS. 1a-d, an OFET can include a dielectric layer (e.g., shown as 8, 8′, 8″, and 8′″), a semiconductor layer (e.g., shown as 6, 6′, 6″, and 6′″), a gate contact (e.g., shown as 10, 10′, 10″, and 10′″), a substrate (e.g., shown as 12, 12′, 12″, and 12′″), and source and drain contacts (e.g., shown as 2, 2′, 2″, 2′″, 4, 4′, 4″, and 4′″).

In certain embodiments, OTFT devices can be fabricated with the present compounds on doped silicon substrates, using SiO₂ as the dielectric, in top-contact geometries. In particular embodiments, the active semiconductor layer which incorporates at least a polymer of the present teachings can be deposited at room temperature or at an elevated temperature. In other embodiments, the active semiconductor layer which incorporates at least one polymer of the present teachings can be applied by spin-coating or printing as described herein. For top-contact devices, metallic contacts can be patterned on top of the films using shadow masks.

In certain embodiments, OTFT devices can be fabricated with the present compounds on plastic foils, using polymers as the dielectric, in top-gate bottom-contact geometries. In particular embodiments, the active semiconducting layer which incorporates at least a polymer of the present teachings can be deposited at room temperature or at an elevated temperature. In other embodiments, the active semiconducting layer which incorporates at least a polymer of the present teachings can be applied by spin-coating or printing as described herein. Gate and source/drain contacts can be made of Au, other metals, or conducting polymers and deposited by vapor-deposition and/or printing.

Other articles of manufacture in which compounds of the present teachings are useful include photovoltaics or solar cells. Compounds of the present teachings can exhibit broad optical absorption and/or a tuned redox properties and bulk carrier mobilities, making them desirable for such applications. Accordingly, the compounds described herein can be used as an acceptor (n-type) semiconductor or a donor (p-type) semiconductor in a photovoltaic design, which includes an adjacent p-type or n-type semiconductor material, respectively, that forms a p-n junction. The compounds can be in the form of a thin film semiconductor, which can be deposited on a substrate to form a composite. Exploitation of compounds of the present teachings in such devices is within the knowledge of a skilled artisan.

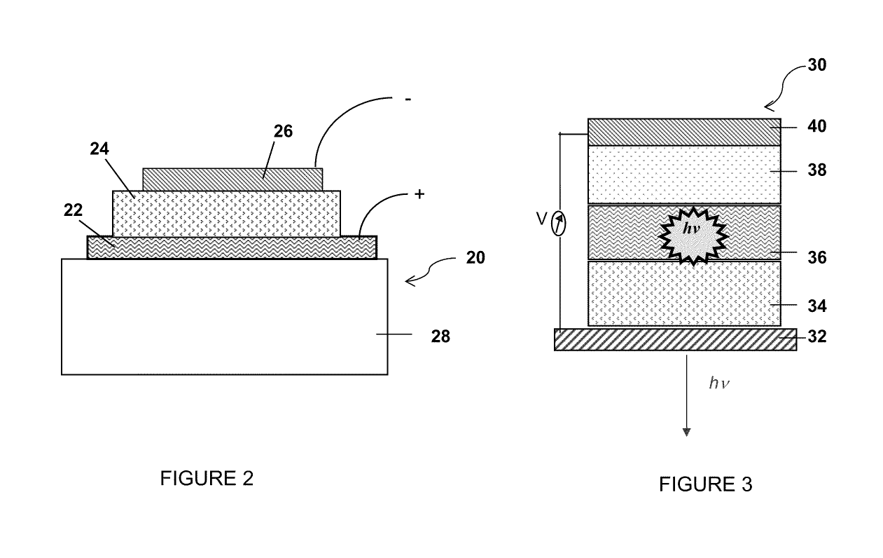

Accordingly, another aspect of the present teachings relates to methods of fabricating an organic light-emitting transistor, an organic light-emitting diode (OLED), or an organic photovoltaic device that incorporates one or more semiconductor materials of the present teachings. FIG. 2 illustrates a representative structure of a bulk-heterojunction organic photovoltaic device (also known as solar cell) 20 which can incorporate one or more compounds of the present teachings as the donor and/or acceptor materials. As shown, a representative solar cell generally includes an anode 22 (e.g., ITO), a cathode 26 (e.g., aluminium or calcium), and an active layer 24 between the anode and the cathode which can incorporate one or more compounds of the present teachings as the electron donor (p-channel) and/or electron acceptor (n-channel) materials on a substrate 28 (e.g., glass). FIG. 3 illustrates a representative structure of an OLED 30 which can incorporate one or more compounds of the present teachings as electron-transporting and/or emissive and/or hole-transporting materials. As shown, an OLED generally includes a substrate (not shown), a transparent anode 32 (e.g., ITO), a cathode 40 (e.g., metal), and one or more organic layers which can incorporate one or more compounds of the present teachings as hole-transporting (n-channel) (layer 34 as shown) and/or emissive (layer 36 as shown) and/or electron-transporting (p-channel) materials (layer 38 as shown).

The following examples are provided to illustrate further and to facilitate the understanding of the present teachings and are not in any way intended to limit the invention.

### Example 1

### Synthesis

All reagents were purchased from commercial sources and used without further purification. All solvents were spectrophotometric grade unless otherwise noted. Mesitylene was distilled over CaH, and stored over molecular sieves. Toluene and non-stabilized HPLC grade dichloromethane were dried using a Glass Contour solvent system. All glassware was flame-dried and kept stringently free from oxygen. Proton nuclear magnetic resonance spectra were recorded on a Varian 500 MHz spectrometer with chemical shifts given in ppm referenced to the solvent. Laser desorption mass spectra were obtained with the Bruker Autoflex III MALDI-TOF spectrometer using polystyrene as an internal standard. Flash chromatography was performed using Sorbent Technologies (Atlanta, Ga.) silica gel.

### Example 1A

### Synthesis of N-(n-octyl)-2,7-bis(phenylethyl)naphthalene-1,8-dicarboximide (2)

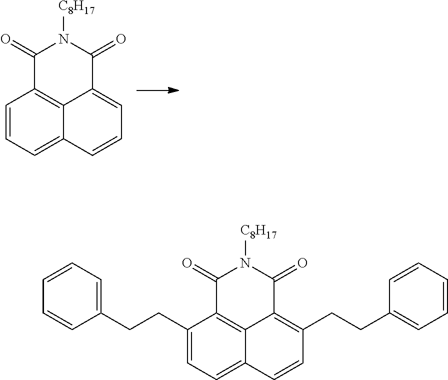

N-(n-octyl)-naphthalene-1,8-dicarboximide (1) (see Hasharoni et al., J. Am. Chem. Soc., 117: 8055-8056 (1995)) (29 mg, 0.094 mmol) and styrene (0.11 mL, 0.96 mmol) were added to an oven-dried flask charged with 3 mL freshly distilled mesitylene. The mixture was thoroughly purged with N₂, after which Ru(H₂)CO(PPh₃)₃ (8 mol %, 7 mg, 0.008 mmol) was added. The solution was heated to 160° C. and set to reflux under N₂ for 24 hours. The flask was opened to air and the solvent driven off, and the product was purified on a silica column using 9:1 hexanes:ethyl acetate as the mobile phase to yield 2 (24 mg, 49%).

¹H NMR (500 MHz, CDCl₃) δ: 7.96 (d, 8.4 Hz, 2H), 7.39 (d, J=8.4 Hz, 2H), 7.32 (m, 8H), 7.21 (m, 2H), 4.23 (t, J=7.7 Hz, 2H), 3.75 (t, J=8.0 Hz, 4H), 3.04 (t, 7.9 Hz, 4H), 1.75 (quintet, J=7.6 Hz, 2H), 1.46 (quintet, J=7.4 Hz, 2H), 1.39 (quintet, J=7.1 Hz, 2H), 1.34 (m, 6H), 0.86 (triplet, J=6.9 Hz, 3H). ¹³C NMR (CDCl₃) δ: 163.0, 148.9, 140.9, 132.1, 130.1, 129.4, 128.6, 127.7, 127.3, 118.2, 39.4, 38.0, 28.4, 28.3, 27.2, 26.3, 21.6, 13.1. HRMS-MALDI-TOF (m/z): [M−H]⁺ calcd for C₃₆H₃₈NO₂, 518.3054. found 518.3079.

### Example 1B

### Synthesis of N,N′-bis(n-octyl)-2,6-bis(phenethyl)naphthalene-1,8:4,5-bis(dicarboximide)/N,N′-bis(n-octyl)-2,7-bis(phenethyl)naphthalene-1,8:4,5-bis(dicarboximide) (4)

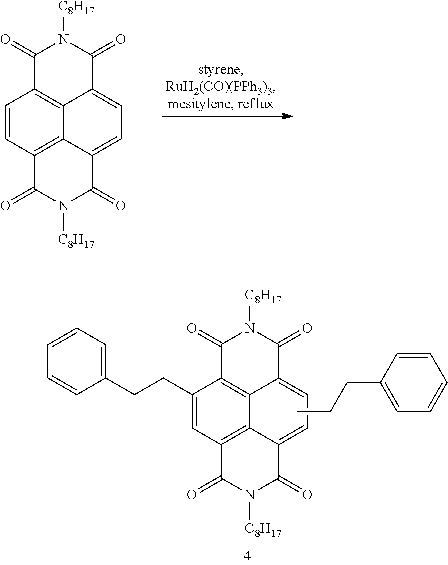

N,N′-bis(n-octyl)-naphthalene-1,8:4,5-bis(dicarboximide) (3) (see Jones et al., Chem. Mater., 19: 2703-2705 (2007)) (250 mg, 0.510 mmol) and styrene (0.59 mL, 5.1 mmol) were added to an oven-dried flask charged with 12 mL freshly distilled mesitylene. The mixture was thoroughly purged with N₂, after which RuH₂(CO)(PPh₃)₃ (5 mol %, 24 mg, 0.026 mmol) was added. The solution was heated to 160° C. and set to reflux under N₂ for 2.5 hours. The flask was opened to air and the solvent driven off. A mixture of 2,6 and 2,7 isomers was isolated with preparatory TLC (69.5:30:0.5 dichloromethane:hexanes:acetone as the eluent), and the reaction yield was estimated at 170 mg (48%). The 2,7-NI isomer was isolated via HPLC.

¹H NMR (CDCl₃) δ: 8.511 (s, 2H); 7.35 (m, 10H); 4.23 (t, J=7.3 Hz, 2H); 4.17 (t, J=7.3 Hz, 2H); 3.86 (t, J=7.5 Hz, 4H); 3.86 (t, J=7.5 Hz, 4H); 1.76 (m, 4H); 1.482 (m, 20H); 0.89 (t, J=6.6 Hz, 6H). ¹³C NMR (CDCl₃) δ: 163.0, 162.9, 150.0, 141.3, 134.8, 129.4, 128.7, 128.5, 126.3, 125.3, 124.5, 123.4, 41.0, 40.9, 39.0, 36.9, 31.9, 31.8, 31.6, 29.4, 29.3, 29.2, 28.1, 27.2, 27.1, 22.7, 14.1. HRMS-MALDI-TOF (m/z): [M+H]⁺ calcd for C₄₆H₅₆N₂O₄, 699.4156. found 699.4188.

### Example 1C

### Synthesis of N-(2′-ethylhexyl)-2,5-bis(phenethyl)perylene-3,4-dicarboximide (6)

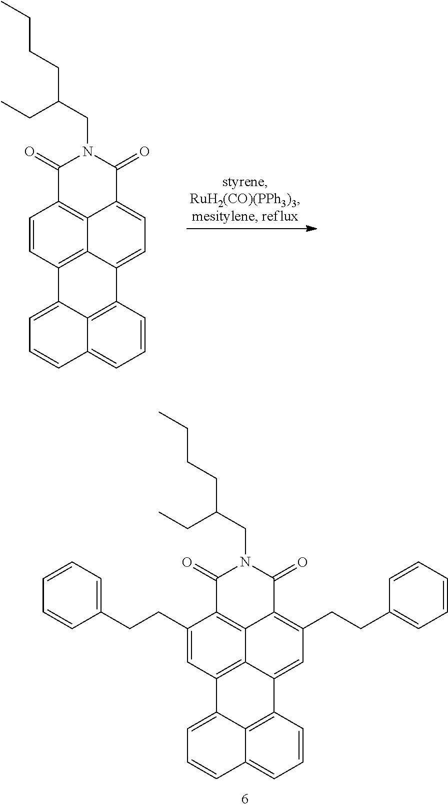

N-(2′-ethylhexyl)-perylene-3,4-dicarboximide (5) (see Feiler et al., Liebigs Annalen, 1229-1244 (1995)) (665 mg, 1.53 mmol) and styrene (3.51 mL, 30.6 mmol) were added to an oven-dried flask charged with 25 mL freshly distilled mesitylene. The mixture was thoroughly purged with N₂, after which RuH₂(CO)(PPh₃)₃ (12 mol %, 168 mg, 0.183 mmol) was added. The solution was heated to 160° C. and set to reflux under N₂ for 48 hours. The flask was opened to air and the solvent driven off, and the product was purified on a silica column using 1:1 dichloromethane:hexanes as the mobile phase to yield 6 (771 mg, 72%).

¹H NMR (500 MHz, CDCl₃) δ: 8.16 (d, J=7.4 Hz, 2H), 7.93 (s, 2H); 7.83 (d, 8 Hz, 2H); 7.55 (t, J=7.8 Hz, 2H); 7.37 (d, J=7.5 Hz, 4H); 7.32 (t, 7.6 Hz, 4H); 7.22 (tot, J=7.2 Hz/1.3 Hz, 2H); 4.23 (m, 2H); 3.75 (t, J=7.9 Hz, 4H); 3.07 (t, 7.9 Hz, 4H); 2.02 (heptet, J=6.3 Hz, 1H); 1.49-1.28 (m, 8H); 0.98 (triplet, J=7.4 Hz, 3H); 0.89 (triplet, J=7.2 Hz, 3H). ¹³C NMR (CDCl₃) δ: 163.8, 148.9, 142.1, 134.7, 133.7, 131.9, 127.3, 130.1, 128.8, 128.5, 128.4, 126.5, 125.9, 124.4, 124.3, 122.8, 117.9, 43.7, 39.6, 37.9, 37.0, 30.8, 28.6, 24.1, 23.3, 14.2, 10.8. HRMS-MALDI-TOF (m/z): [M−H]⁺ calcd for C₄₆H₄₁NO₂, 642.3367. found 642.3389.

### Example 1D

### Synthesis of N,N′-bis(n-octyl)-2,5,8,11-tetrakis(phenethyl)perylene-3, 4:9,10-bis(dicarboximide) (8)

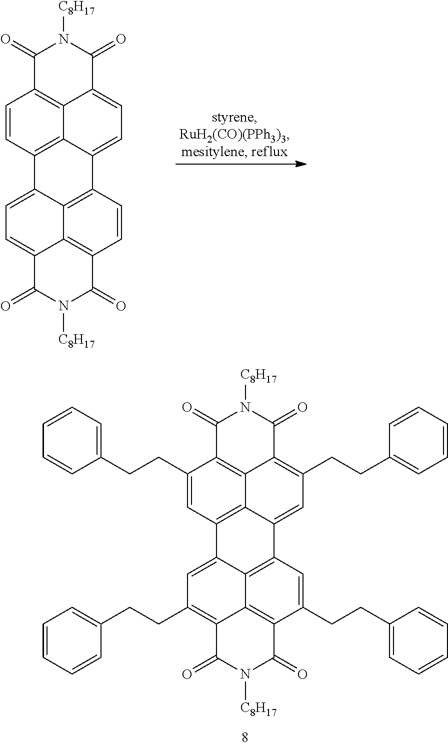

N,N′-bis(n-octyl)-perylene-3, 4:9,10-bis(dicarboximide) (7) (see Jones et al., Angew. Chem., Int. Ed., 43: 6363-6366 (2004)) (500 mg, 0.814 mmol) and styrene (1.85 mL, 16.1 mmol) were added to an oven-dried flask charged with 25 mL freshly distilled mesitylene. The mixture was thoroughly purged with N₂, after which RuH₂(CO)(PPh₃)₃ (12 mol %, 100 mg, 0.109 mmol) was added. The solution was heated to 160° C. and set to reflux under N₂ for 72 hours. The flask was opened to air and the solvent driven off, and the product was powdered out from dichloromethane. The mother liquor was purified on a silica column using chloroform as the mobile phase to yield 8 (625 mg, 75%).

¹H NMR (CD₂Cl₂) δ: 7.81 (s, 4H); 7.37-7.32 (m, 16H); 7.23 (m, 4H); 4.21 (m, 4H); 3.75 (t, J=7.8 Hz, 8H); 3.08 (t, J=7.8 Hz, 8H); 1.77 (quintet, J=7.5 Hz, 4H); 1.51-1.25 (m, 20H); 0.88 (t, J=6.9 Hz, 6H). ¹³C NMR (CDCl₃) δ: 163.4, 149.1, 141.7, 132.4, 131.5, 127.5, 126.2, 124.2, 120.0, 40.7, 39.1, 36.8, 31.9, 29.5, 29.4, 28.2, 27.4, 22.7, 14.2. HRMS-MALDI-TOF (m/z): [M−1]⁺ calcd for C₇₂H₇₃N₂O₄, 1029.5570. found 1029.5724.

### Example 1E

### Synthesis of N,N′-bis(2-ethylhexyl)-2,5,10,13-tetrakis(phenethyl)terrylene-3, 4:11,12-bis(dicarboximide) (10)

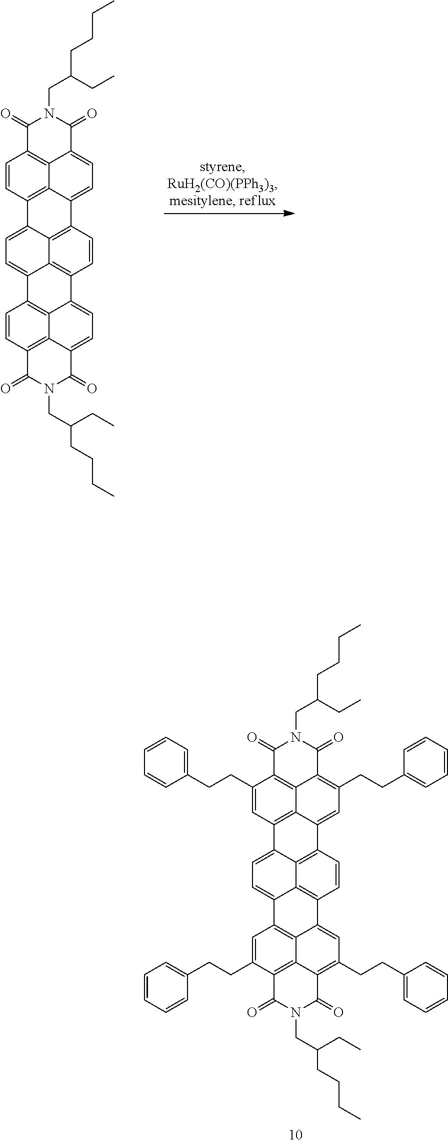

N,N′-bis(2-ethylhexyl)-terrylene-3,4:11,12-bis(dicarboximide) (9) (see Nolde et al., Chem. Eur. J., 11: 3959-3967 (2005)) (0.03 g, 0.041 mmol) and styrene (0.812 mmol, 0.093 mL) were added to a flame-dried flask charged with freshly distilled mesitylene (2 mL). The mixture was purged with N₂, after which RuH₂(CO)(PPh₃)₃ (12 mol %, 4.5 mg, 0.005 mmol) was added. The mixture was brought to reflux for 48 hours, then the mesitylene was blown down with N₂. The crude mixture was purified on a silica column eluted with dichloromethane to yield pure 10 (0.038 g, 81% yield) as a blue powder.

¹H NMR δ (CDCl₃): 8.18 (s, 4H); 7.97 (s, 4H); 7.38 (m, 20H); 4.22 (m, 2H); 3.82 (t, J=7.2 Hz, 8H); 3.14 (t, J=7.2 Hz, 8H); 2.02 (m, 4H); 1.57 (m, 22H); 1.00 (m, 3H); 0.91 (m, 3H). ¹³C NMR (CDCl₃) δ: 164.4, 145.8, 135.9, 135.5, 131.7, 131.0, 129.2, 129.0, 128.5, 126.3, 126.0, 124.4, 123.9, 122.1, 43.7, 39.6, 37.9, 37.0, 30.8, 28.6, 24.1, 23.3, 14.2, 10.8. HRMS-MALDI-TOF (m/z): [M]⁺ calcd for C₈₂H₇₈N₂O₄, 1154.5962. found 1154.5976.

### Example 1F

### Synthesis of Additional Compounds (12, 14, 16, 18, 20, 22, 24, and 26)

Additional compounds (12, 14, 16, 18, 20, 22, 24, and 26) were prepared according to the procedures analogous to those described in Examples 1A-1E. The starting reagent, the reaction time, and yield are summarized in Table 1.

### Example 2

### Characterization of Compounds

### Example 2A

### Electrochemistry

Electrochemical measurements were performed using a CH Instruments Model 622 electrochemical workstation. Measurements for compounds 4, 6, 8, and 10 were performed in dichloromethane containing 0.1 M tetrabutylammonium hexafluorophosphate (TBAPF₆) electrolyte. Measurements for 2 were performed in benzonitrile with the same electrolyte. A 1.0 mm diameter platinum disk electrode, platinum wire electrode, and Ag/AgO⁺ reference electrode were employed. The ferrocene/ferrocenium redox couple (Fc/Fc⁺, 0.46 V vs. SCE) (see Connelly et al., Chem. Rev., 96: 877-910 (1996)) was used as an internal standard. TBAPF₆ was recrystallized twice from ethyl acetate prior to use.

Alkylation (e.g., substitution of the phenethyl groups) ortho to the imide nitrogen atoms appears to render the present compounds somewhat more difficult to reduce, but easier to oxidize (Table 3). The reduction potentials of the disubstituted compounds (2, 4, and 6) are approximately 130 mV more negative than those of their parent compounds. Correspondingly, the oxidation potential of the disubstituted 6 is 40 mV less positive than that of 6. Tetrasubstituted 8 and 10 have reduction potentials that are more negative by ˜200 mV than their unsubstituted analogs. These shifts in redox potentials reasonably arise from the electron-donating nature of the alkylating (e.g., phenethyl) groups. The oxidation and reduction potentials of all alkylated molecules shift in the same direction by nearly the same amount, such that the corresponding HOMO-LUMO energy gap remains the same, as is reflected in their electronic absorption and emission spectra described below.

TABLE 3

| Chromophore | E⁺_{1/2} | E⁻_{1/2} | E²⁻_{1/2} |
|---|---|---|---|
| N-(n-octyl)-naphthalene-1,8-dicarboximide (1) | — | −1.40^{a} | — |
| N-(n-octyl)-2,7-bis(phenylethyl)-naphthalene-1,8- | — | −1.53^{b} | — |
| dicarboximide (2) | | | |
| N,N′-bis(n-octyl)-naphthalene-1,8: 4,5- | — | −0.48^{a} | −0.99^{a} |
| bis(dicarboximide) (3) | | | |
| N,N′-bis(n-octyl)-2,7-bis(phenylethyl)-naphthalene- | — | −0.61 | −1.15 |
| 1,8: 4,5-bis(dicarboximide) (4) | | | |
| N-(2′-ethylhexyl)-perylene-3,4-dicarboximide (5) | 1.41 | −1.00^{c} | −1.49^{c} |
| N-(2′-ethylhexyl)-bis(2,7-phenylethyl)-perylene-3,4- | 1.37 | −1.13 | −1.43 |
| dicarboximide (6) | | | |
| N,N′-bis(n-octyl)-perylene-3,4: 9,10- | 1.67 | −0.50^{c} | −0.73^{c} |
| bis(dicarboximide) (7) | | | |
| N,N′-bis(n-octyl)-2,5,8,11-tetrakis(phenylethyl)- | 1.63 | −0.75 | −0.93 |
| perylene-3,4: 9,10-bis(dicarboximide) (8) | | | |
| N,N′-bis(2-ethylhexyl)-terrylene-3,4: 11,12- | 1.12 | −0.63^{c} | — |
| bis(dicarboximide) (9) | | | |
| N,N′-bis(2-ethylhexyl)-2,5,10,13-tetrakis(phenylethyl)- | 1.10 | −0.83 | — |
| terrylene-3,4: 11,12-bis(dicarboximide) (10) | | | |

^{a}Gosztola et al., J. Phys. Chem. A, 104: 6545-6551 (2000);
^{b}obtained in benzonitrile with 0.1 M TBAPF₆;
^{c}Lee et al., J. Am. Chem. Soc., 121: 3513-3520 (1999).

### Example 2B

### Optical Spectroscopy

Steady-state electronic absorption spectra were recorded on a Shimadzu 1601 UV/Vis spectrometer with a 2 mm quartz cuvette. Fluorescence measurements were performed using a PTI Quanta-Master 1 single photon counting spectrofluorimeter in a right angle configuration with a 1 cm quartz cuvette.

Femtosecond transient absorption measurements were made using a Ti:sapphire laser system as detailed in previously reported work. See Kelley et al., J. Am. Chem. Soc., 129: 3173-3181 (2007). The wavelengths used to excite the samples were 390 nm (1-4), 416 nm (5-8), and 650 nm (9 and 10). The instrument response function for the pump-probe experiments is 160 fs. The transient spectra were obtained using 10 s of averaging at a given delay time. Glass cuvettes with a 2 mm pathlength were used and the samples were dissolved in dry toluene and irradiated with 1.0 μJ pulses at the excitation wavelength. Analysis of the kinetic data was performed at multiple wavelengths using a Levenberg-Marquardt nonlinear least-squares fit to a general sum-of-exponentials function with an added Gaussian to account for the finite instrument response.

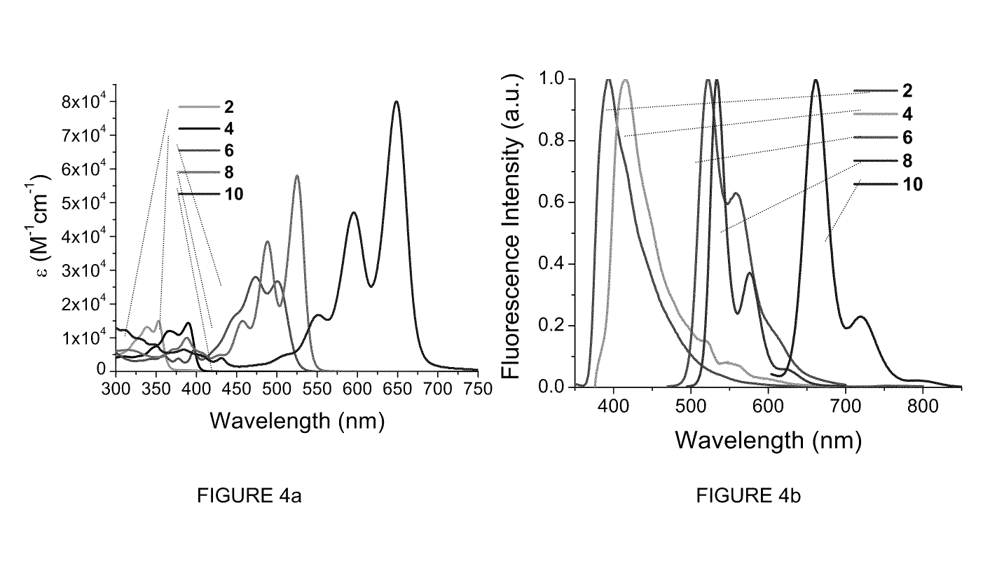

The ground state electronic absorption spectra for 2, 4, 6, 8, and 10 are shown in FIG. 4a. The intensity of the S₀→>S₁ transition increases with the size of the aromatic core, while the energy decreases. The fluorescence spectrum of each chromophore is shown in FIG. 4b and summarized in Table 4 along with that of compounds 1, 3, 5, 7, and 9. A similar trend with the emission maxima moving to longer wavelengths as the size of the aromatic core increases was observed. The electronic absorption and fluorescence properties of all the alkylated chromophores are very similar to those of the parent compounds. No observable trend is apparent for the slight shifts in λₘₐₓ; however, with the exception of 2, all alkylated compounds have slightly lower extinction coefficients than their parent compounds. The fluorescence emission maxima of the alkylated compounds (Table 4) are similar to those of the parent compounds as reported in the literature. See Hydlovic et al., Phtochem. Photobiol. A., 112: 197-203 (1998); Licchelli et al., Chem. Eur. J., 8: 5161-5169 (2002); Weil et al., Chem. Eur. J., 8: 4742-4750 (2002); and Sadrai et al., J. Phys. Chem., 96: 7988-7996 (1992). However, the phenethyl groups significantly reduce the fluorescence quantum yields of only the naphthalene derivatives 2 and 4, while the yields for the higher rylenes are unaffected.

TABLE 4

| | λₘₐₓ (nm) (ε | λₑₘ | E(S₁)^{c} | |
|---|---|---|---|---|
| Chromophore | (M⁻¹ cm⁻¹)) | (nm) | (eV) | φ_{F} |
| 1 | 350 (10647)^{a} | 386^{a} | 3.38 | 0.25^{d} |
| 2 | 353 (15000) | 394 | 3.34 | 0.08 |
| 3 | 382 (14500)^{a} | 407^{a} | 3.15 | 0.0018^{e} |
| 4 | 390 (14000) | 415 | 3.09 | 0.0005 |
| 5 | 505 (29970)^{a} | 539^{a} | 2.38 | 0.98^{f} |
| 6 | 501 (27000) | 521 | 2.43 | 0.98 |
| 7 | 526 (80000)^{a} | 535^{a} | 2.34 | 0.99^{g} |
| 8 | 525 (58000) | 533 | 2.35 | 0.99 |
| 9 | 650 (93000)^{b} | 673^{b} | 1.88 | 0.95^{h} |
| 10 | 649 (80000) | 661 | 1.90 | 0.95 |

^{a}Gosztola et al., J. Phys. Chem. A, 104: 6545-6551 (2000);
^{b}Holtrup et al., Chem. Eur. J., 3: 219-225 (1997);
^{c}Determined from the average energy of the absorption and emission maxima;
^{d}Licchelli et al., Chem. Eur. J., 8: 5161-5169 (2002);
^{e}Weil et al., Chem. Eur. J., 8: 4742-4750 (2002); and
^{f}Sadrai et al., J. Phys. Chem., 96: 7988-7996 (1992)benzonitrile with 0.1M TBAPF₆; and
^{g}Weil et al., Chem. Eur. J., 8: 4742-4750 (2002).

The transient absorption spectra of naphthalene monoimide 2, specifically, the S₁→>Sₙ absorption is characterized by a single broad band at 525 nm, which decays monoexponentially with a time constant τ_{D}=34 ps. This is similar to the time constant of other NMI compounds and is attributed to rapid intersystem crossing leading to the triplet state of 2. Naphthalene diimide 4 displays an initial transient spectrum with a maximum at 575 nm, which blue-shifts substantially to 530 nm with a 4 ps time constant. The residual 530 nm absorption band decays further with a 175 ps time constant. Without wishing to be bound by any particular theory, these processes may be attributed to the rapid formation of an exciplex involving ¹*NI and one of the appended phenyl groups, and its subsequent slower relaxation. Naphthalene diimide derivatives have been shown to oxidize phenyl rings covalently bonded through the imide position due to their high excited state energies. A recent report has demonstrated that electron transfer via the 2-position on NI is 1000-fold faster than through the imide. See Chaignon et al., Chemical Communications (Cambridge, England), page 64-66 (2007). Accordingly, it is conceivable that exciplex formation is occurring on the picosecond timescale or faster. The transient absorption spectra and monoexponential Sᵢ lifetimes of the perylene derivatives 6 and 8 are similar to those reported earlier for other PMI and PDI derivatives (see Hayes et al., J. Am. Chem. Soc., 122: 5563-5567 (2000) and Giaimo et al., J. Phys. Chem. A, 112: 2322-2330 (2008)) and are illustrative of the minimal perturbation that the four alkyl substituents have on the electronic structures of these molecules (Table 5). The transient absorption spectrum of a terrylene diimide has not been reported previously. The bleaching of the ground state absorption bands is accompanied by strong stimulated emission features at 725 nm, as well as weak positive absorption changes at 400-540 nm and 765-800 nm. All of these transient spectral changes decrease with a monoexponential decay time of τ_{D}=3.8 ns, which is very similar to that observed for the corresponding perylene derivatives 6 and 8.

TABLE 5

| Chromophore | τ_{D} (ps) |
|---|---|
| 1 | <50^{a} |
| 2 | 34 ± 1 |
| 3 | <20^{a} |
| 4 | 4.0 ± 0.3 |
| | 175 ± 10 |
| 5 | 3500 ± 100^{a} |
| 6 | 4700 ± 88 |

^{a}Hayes et al., J. Am. Chem. Soc., 122: 5563-5567 (2000);
^{b}Schweitzer et al., J. Phys. Chem., 107: 3199-3207 (2003).

### Example 2C

### EPR and ENDOR Spectroscopy

Continuous wave electron paramagnetic resonance (EPR) and electron nuclear double resonance (ENDOR) spectra were acquired with a Bruker Elexsys E580 spectrometer, fitted with the DICE ENDOR accessory, EN801 resonator, and an ENI A-500 RF power amplifier. RF powers ranged from 240-400 W across the 7 MHz scanned range, and microwave power was 2-6 mW. The sample temperature of 290 K was controlled by a liquid nitrogen flow system. All samples and solvents were handled in a nitrogen atmosphere glovebox (MBraun Unilab). Samples were prepared in DCM with 2% triethylamine (TEA) (w/w) and loaded into 1.4 mm I.D. quartz tubes which were sealed with a 0.5-1.0 cm plug of vacuum grease and wrapped tightly with Parafilm. TEA was dried with CaH₂ and filtered through dry silica gel prior to use and storage in the glovebox. Photochemical reduction to form monoanions was accomplished by exciting the sample with an Ar⁺ laser (514.5 nm, 40 mW) beam elongated in one dimension with a cylindrical lens. In all cases the photochemical reductions using TEA formed solely monoanions of the PDI oligomers as monitored by UV-Vis. UV-Vis spectra acquired through the quartz tube match the spectra of PDI radical anions generated electrochemically. A spline fit baseline correction was applied to the ENDOR spectra following integration.

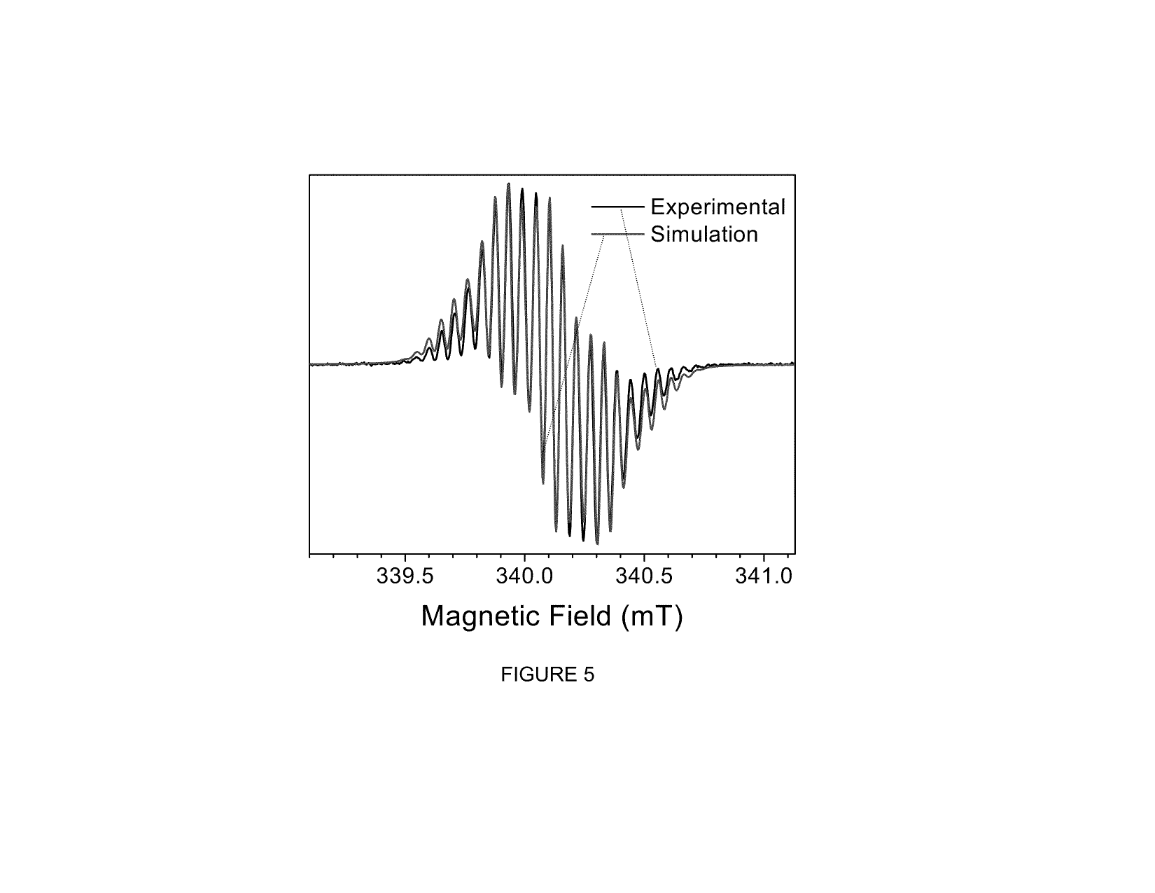

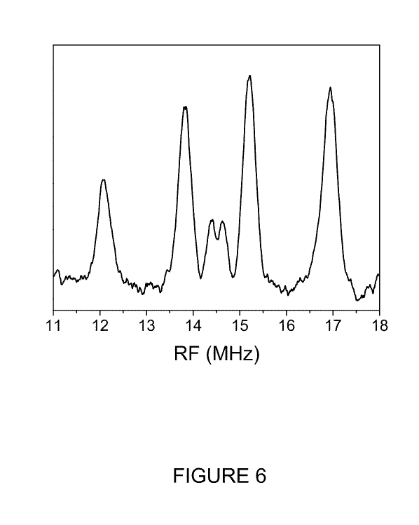

The EPR spectrum of 8^{•-} (FIG. 5) is inhomogeneously broadened due to the large number of hyperfine coupling constants (hfcc's). Isotropic hfcc's were obtained from ENDOR spectroscopy in liquid DCM at the ENDOR resonance condition v_{ENDOR}^{±}=|vₙ±a_{H}/2| where v_{ENDOR}^{±} are the ENDOR transition frequencies and vₙ is the proton Larmor frequency. The ENDOR spectrum of 8^{•-} obtained at 290 K is presented in FIG. 6 and exhibits three line pairs with hfcc's of 4.9, 1.4, and 0.2 MHz. The largest hfcc has been assigned to that of the bay-region protons (positions 1, 6, 7, and 12) on the perylene core and is similar in magnitude to previously reported PDI derivatives. See Tauber et al., J. Am. Chem. Soc., 128: 1782-1783 (2006) and Wilson et al., J. Am. Chem. Soc., 131: 8952-8957 (2009). The two smaller proton hfcc's can be attributed to the β-protons on the phenethyl groups. Without wishing to be bound by any particular theory, the 0.2 MHz splitting also could be due to the octyl group attached to the imide nitrogen atom. These assignments were confirmed using unrestricted DFT to calculate the hfcc's using B3LYP funtionals with the expanded double-ζ EPR-II basis set. The computed proton hfcc's were 5.4 (perylene protons 1, 6, 7, 12), 1.6 (β-phenethyl protons), and 0.3 (β-phenethyl and β-octyl protons) MHz and 1.7 MHz for the nitrogens. A simulation of the EPR spectrum of 8^{•-} (Winsim) (see Duling, D. R., J. Magn. Res. B., 104: 105-110 (1994) using the ENDOR results to lock the hfcc values allows determination of the nitrogen hfcc to be 1.4 MHz, which also is in reasonable agreement with the DFT calculation. The EPR and ENDOR spectra reveal that the spin (and charge) distribution within the radical ion of PDI is very similar to that of PDI, so that one may anticipate that applications of these molecules as electron acceptors would result in similar behavior to that of the parent molecule except for being somewhat more difficult to reduce.

### Example 2D

### Density Function Theory (DFT) Calculations

The structure of a model PDI radical anion with ethyl substituents at the 2, 5, 8, and 11 positions, as well as at the imide positions, was first optimized using unrestricted DFT with Q-Chem 3.1 at the B3LYP/6-31 G* level. A single-point unrestricted DFT calculation at this optimized geometry was performed to calculate the isotropic hyperfine coupling constants (hfcc's) using a B3LYP functional with the expanded double-ζ EPR-II basis set (see Improta et al., Chem. Rev., 104: 1231-1253 (2004)) and Gaussian 98W.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

1. A compound of formula I: 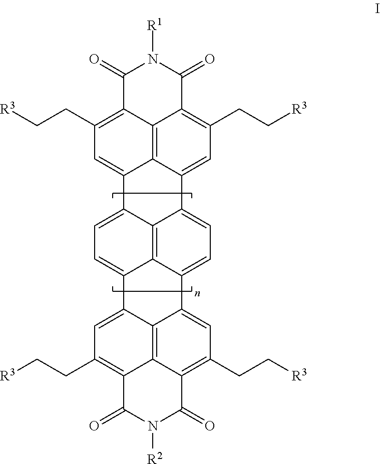
wherein:
R¹ and R² independently are selected from H, a C₁₋₄₀ alkyl group, a C₂₋₄₀ alkenyl group, a C₁₋₄₀ haloalkyl group, and an organic group comprising 1-4 cyclic moieties,
wherein:
each of the C₁₋₄₀ alkyl group, the C₂₋₄₀ alkenyl group, and the C₁₋₄₀ haloalkyl group optionally is substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group is bonded either directly or via an optional linker to the imide nitrogen; and
each of the 1-4 cyclic moieties in the organic group is the same or different, is bonded either directly or via an optional linker to each other, and optionally is substituted with 1-5 substituents independently selected from a halogen, oxo, —CN, NO₂, OH, ═C(CN)₂, —NH₂, —NH(C₁₋₄₀ alkyl), —N(C₁₋₄₀ alkyl)₂, —S(O)₂OH, —CHO, —C(O)OH, —C(O)—C₁₋₄₀ alkyl, -sC(O)—OC₁₋₄₀ alkyl, —C(O)NH₂, —C(O)NH—C₁₋₄₀ alkyl, —C(O)N(C₁₋₄₀ alkyl)₂, —SiH₃, —SiH(C₁₋₄₀ alkyl)₂, —SiH₂(C₁₋₄₀ alkyl), —Si(C₁₋₄₀ alkyl)₃, —O—C₁₋₄₀ alkyl, —O—C₁₋₄₀ alkenyl, —O—C₁₋₄₀ haloalkyl, a C₁₋₄₀ alkyl group, a C₁₋₄₀ alkenyl group, and a C₁₋₄₀ haloalkyl group; and
R³ is selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 membered heteroaryl group, the C₃₋₁₄ cycloalkyl group, and the 3-14 membered cycloheteroalkyl group optionally is substituted with 1-10 substituents independently selected from a halogen, —CN, a C₁₋₁₀ alkyl group, a C₁₋₁₀ alkoxy group, and a C₁₋₁₀ haloalkyl group; and
n is 0, 1, or 2.

2. The compound of claim 1, wherein R³ is selected from Si(CH₃), Si(OCH₃)₃, a cyclohexyl group, a C₁₋₁₀ alkyl group, a C₁₋₁₀ haloalkyl group, and a phenyl group optionally substituted with 1-5 substituents independently selected from a halogen, a C₁₋₆ alkyl group, a C₁₋₆ alkoxy group, and a C₁₋₆ haloalkyl group.

3. The compound of claim 1, wherein R¹ and R² independently are selected from H, -L-R^{a}, -L′-Cy¹, -L′-Cy¹-L′-Cy², -L′-Cy¹-L′-Cy²-Cy², -L′-Cy¹-Cy¹, -L′-Cy¹-Cy¹-L′-Cy², -L′-Cy¹-Cy¹-L′-Cy²-Cy², -L′-Cy¹-L-R^{a}, -L′-Cy¹-L′-Cy²-L-R^{a}; -L′-Cy¹-L′-Cy²-Cy²-L-R^{a}, -L′-Cy¹-Cy¹-L-R^{a}, and -L′-Cy¹-Cy¹-L′-Cy²-L-R^{a};
wherein:
Cy¹ and Cy² independently are selected from a C₆₋₁₄ aryl group, a 5-14 membered heteroaryl group, a C₃₋₁₄ cycloalkyl group, and a 3-14 membered cycloheteroalkyl group, each of which optionally can be substituted with 1-5 substituents independently selected from a halogen, —CN, oxo, ═C(CN)₂, a C₁₋₆ alkyl group, a C₁₋₆ alkoxy group, and a C₁₋₆ haloalkyl group;
L, at each occurrence, independently is a covalent bond or a linker selected from —Y—O—Y—, —Y—[S(O)_{w}]—Y—, —Y—C(O)—Y—, —Y—[NR^{c}C(O)]—Y—, —Y—[C(O)NR^{c}]—, —Y—NR^{c}—, —Y—[SiR^{c}₂]—Y—, a divalent C₁₋₄₀ alkyl group, a divalent C₂₋₄₀ alkenyl group, and a divalent C₁₋₄₀ haloalkyl group;
L′, at each occurrence, independently is a covalent bond or a linker selected from —Y—O—Y—, —Y—[S(O)_{w}]—Y—, —Y—C(O)—Y—, —Y—[NR^{c}(O)]—Y—, —Y—[C(O)NR^{c}]—, —Y—NR^{c}—Y—, —Y—[SiR^{c}₂]—Y—, a divalent C₁₋₄₀ alkyl group, a divalent C₂₋₄₀ alkenyl group, and a divalent C₁₋₄₀ haloalkyl group;
wherein:
Y, at each occurrence, independently is selected from a divalent C₁₋₄₀ alkyl group, a divalent C₂₋₄₀ alkenyl group, a divalent C₁₋₄₀ haloalkyl group, and a covalent bond;
R^{c} is selected from H, a C₁₋₆ alkyl group, a C₆₋₁₄ aryl group, and a —C₁₋₆ alkyl-C₆₋₁₄ aryl group; and
w is 0, 1, or 2; and
R^{a} is selected from a C₁₋₄₀ alkyl group, a C₂₋₄₀ alkenyl group, and a C₁₋₄₀ haloalkyl group, each of which optionally can be substituted with 1-10 substituents independently selected from a halogen, —CN, NO₂, OH, —NH₂, —NH(C₁₋₂₀ alkyl), —N(C₁₋₂₀ alkyl)₂, —S(O)₂OH, —CHO, —C(O)—C₁₋₂₀ alkyl, —C(O)OH, —C(O)—OC₁₋₂₀ alkyl, —C(O)NH₂, —C(O)NH—C₁₋₂₀ alkyl, —C(O)N(C₁₋₂₀ alkyl)₂, —OC₁₋₂₀ alkyl, —SiH₃, —SiH(C₁₋₂₀ alkyl)₂, —SiH₂(C₁₋₂₀ alkyl), and —Si(C₁₋₂₀ alkyl)₃.

4. The compound of claim 1, wherein R¹ and R² independently are selected from a linear or branched C₃₋₄₀ alkyl group, a linear or branched C₃₋₄₀ alkenyl group, —Y-phenyl and —Y-cyclohexyl, wherein Y is a divalent C₁₋₁₀ alkyl group or a covalent bond, and the phenyl group and the cyclohexyl group optionally are substituted with 1-5 substituents independently selected from a halogen, a C₁₋₆ alkyl group, a C₁₋₆ alkoxy group, and a C₁₋₆ haloalkyl group.

5. The compound of claim 1, wherein R¹ and R² independently are a linear or branched C₃₋₄₀ alkyl or C₃₋₄₀ alkenyl group.

6. The compound of claim 1, wherein the compound has the formula: 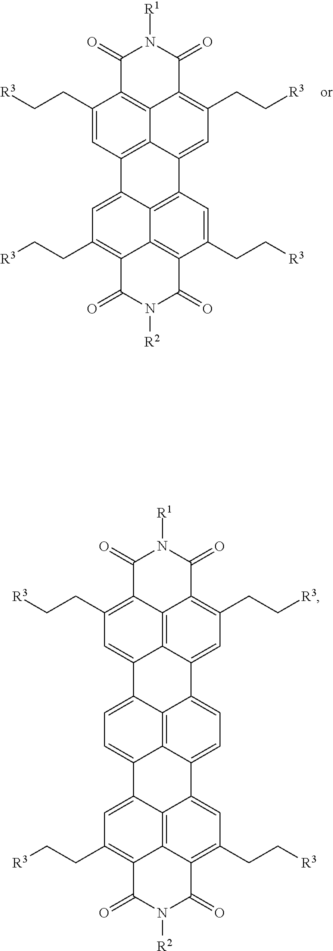
wherein
R¹ and R² independently are selected from a linear or branched C₃₋₄₀ alkyl group, a linear or branched C₃₋₄₀ alkenyl group, and a linear or branched C₃₋₄₀ haloalkyl group; and
each R³ is selected from Si(CH₃)₃, Si(OCH₃)₃, a cyclohexyl group, a C₁₋₁₀ alkyl group, a C₁₋₁₀ haloalkyl group, and a phenyl group optionally substituted with 1-5 substituents independently selected from a halogen, a C₁₋₆ alkyl group, a C₁₋₆ alkoxy group, and a C₁₋₆ haloalkyl group.

7. An electronic, opto-electronic, or optical device comprising a semiconductor component comprising the compound of claim 1.

8. The device of claim 7, wherein the device is selected from a thin film transistor device, an organic light-emitting transistor, and an organic photovoltaic device.

9. An electronic, opto-electronic, or optical device comprising a composite, wherein the composite comprises a dielectric material in contact with a thin film semiconductor comprising the compound of claim 1.

10. An electronic, opto-electronic, or optical device comprising a composite, wherein the composite comprises a dielectric material in contact with a thin film semiconductor comprising the compound of claim 6.

11. A method of preparing the compound of claim 1, the method comprising: reacting a compound of formula I′: 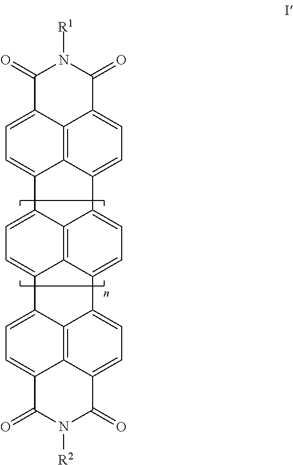
with a compound of the formula III: 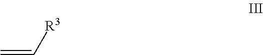
in the presence of a ruthenium catalyst,
wherein R¹, R², R³, and n are as defined in claim 1.

12. The method of claim 11, wherein the ruthenium catalyst is Ru(H₂)CO(PPh₃)₃.

13. The method of claim 11, wherein reacting the compounds is carried out in mesitylene.

14. The method of claim 11, wherein reacting the compounds is carried out at an elevated temperature.

15. The method of claim 11, wherein the compound of formula I′ is reacted with an excess amount of the compound of formula III.

16. A compound of formula II: 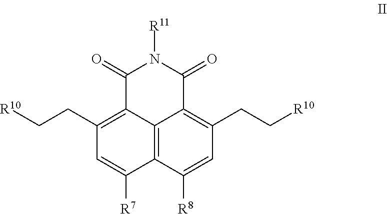
wherein:
R⁷ and R⁸ independently are selected from H, halogen, —CN, a C₁₋₁₀ alkyl group, a C₁₋₁₀ alkoxy group, and a C₁₋₁₀ haloalkyl group; or alternatively,
R⁷ and R⁸ together can be: 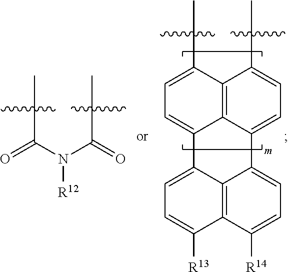
R¹⁰ is selected from SiR₃, SiOR₃, a C₁₋₄₀ alkyl group, a C₁₋₄₀ haloalkyl group, a C₆₋₁₄ aryl group, a 5-14 membered heteroaryl group, a C₃₋₁₄ cycloalkyl group, and a 3-14 membered cycloheteroalkyl group, wherein R, at each occurrence, independently is selected from a C₁₋₄₀ alkyl group and a C₁₋₄₀ haloalkyl group, and each of the C₆₋₁₄ aryl group, the 5-14 membered heteroaryl group, the C₃₋₁₄ cycloalkyl group, and the 3-14 membered cycloheteroalkyl group optionally can be substituted with 1-10 substituents independently selected from a halogen, —CN, a C₁₋₁₀ alkyl group, a C₁₋₁₀ alkoxy group, and a C₁₋₁₀ haloalkyl group;
R¹¹ and R¹² independently are selected from H, a C₁₋₄₀ alkyl group, a C₂₋₄₀ alkenyl group, a C₁₋₄₀ haloalkyl group, and an organic group comprising 1-4 cyclic moieties,
wherein:
each of the C₁₋₄₀ alkyl group, the C₂₋₄₀ alkenyl group, and the C₁₋₄₀ haloalkyl group optionally is substituted with 1-10 substituents independently selected from a halogen, —CN, NO₂, OH, —NH₂, —NH(C₁₋₄₀ alkyl), —N(C₁₋₄₀ alkyl)₂, —S(O)₂OH, —CHO, —C(O)—C₁₋₄₀ alkyl, —C(O)OH, —C(O)—OC₁₋₄₀ alkyl, —C(O)NH₂, —C(O)NH—C₁₋₄₀ alkyl, —C(O)N(C₁₋₄₀ alkyl)₂, —OC₁₋₄₀ alkyl, —SiH₃, —SiH(C₁₋₄₀ alkyl)₂, —SiH₂(C₁₋₄₀ alkyl), and —Si(C₁₋₄₀ alkyl)₃;
each of the C₁₋₄₀ alkyl group, the C₂₋₄₀ alkenyl group, the C₁₋₄₀ haloalkyl group, and the organic group is bonded either directly or via an optional linker to the imide nitrogen atom; and
each of the 1-4 cyclic moieties in the organic group is the same or different, is bonded either directly or via an optional linker to each other, and optionally is substituted with 1-5 substituents independently selected from a halogen, oxo, —CN, NO₂, OH, ═C(CN)₂, —NH₂, —NH(C₁₋₄₀ alkyl), —N(C₁₋₄₀ alkyl)₂, —S(O)₂OH, —CHO, —C(O)OH, —C(O)—C₁₋₄₀ alkyl, —C(O)—OC₁₋₄₀ alkyl, —C(O)NH₂, —C(O)NH—C₁₋₄₀ alkyl, —C(O)N(C₁₋₄₀ alkyl)₂, —SiH₃, —SiH(C₁₋₄₀ alkyl)₂, —SiH₂(C₁₋₄₀ alkyl), —Si(C₁₋₄₀ alkyl)₃, —O—C₁₋₄₀ alkyl, —O—C₁₋₄₀ alkenyl, —O—C₁₋₄₀ haloalkyl, a C₁₋₄₀ alkyl group, a C₁₋₄₀ alkenyl group, and a C₁₋₄₀ haloalkyl group;
R¹³ and R¹⁴ independently are selected from H, halogen, —CN, a C₁₋₁₀ alkyl group, a C₁₋₁₀ alkoxy group, and a C₁₋₁₀ haloalkyl group; and
m is 0, 1, 2, or 3.

17. The compound of claim 16, wherein R¹⁰ is selected from Si(CH₃)₃, Si(OCH₃)₃, a cyclohexyl group, a C₁₋₁₀ alkyl group, a C₁₋₁₀ haloalkyl group, and a phenyl group optionally substituted with 1-5 substituents independently selected from a halogen, a C₁₋₆ alkyl group, a C₁₋₆ alkoxy group, and a C₁₋₆ haloalkyl group.

18. The compound of claim 16, wherein R¹¹ and R¹² independently are selected from H, -L-R^{a}, -L′-Cy¹, -L′-Cy¹-L′-Cy², -L′-Cy¹-L′-Cy²-Cy², -L′-Cy¹-Cy¹, -L′-Cy¹-Cy¹-L′-Cy², -L′-Cy¹-Cy¹-L′-Cy²-Cy², -L′-Cy¹-L-R^{a}, -L′-Cy¹-L′-Cy²-L-R^{a}, -L′-Cy¹-L′-Cy²-Cy²-L-R^{a}, -L′-Cy¹-Cy¹-L-R^{a}, and -L′-Cy¹-Cy¹-L′-Cy²-L-R^{a};
wherein:
Cy¹ and Cy² independently are selected from a C₆₋₁₄ aryl group, a 5-14 membered heteroaryl group, a C₃₋₁₄ cycloalkyl group, and a 3-14 membered cycloheteroalkyl group, each of which optionally can be substituted with 1-5 substituents independently selected from a halogen, —CN, oxo, ═C(CN)₂, a C₁₋₆ alkyl group, a C₁₋₆ alkoxy group, and a C₁₋₆ haloalkyl group;
L, at each occurrence, independently is a covalent bond or a linker selected from —Y—O—Y—, —Y—[S(O)_{w}]—Y—, —Y—C(O)—Y—, —Y—[NR^{c}(O)]—Y—, —Y—[C(O)NR^{c}]—, —Y—NR^{c}—, —Y—[SiR^{c}₂]—Y—, a divalent C₁₋₄₀ alkyl group, a divalent C₂₋₄₀ alkenyl group, and a divalent C₁₋₄₀ haloalkyl group;
L′, at each occurrence, independently is a covalent bond or a linker selected from —Y—O—Y—, —Y—[S(O)_{w}]—Y—, —Y—C(O)—Y—, —Y—[NR^{c}C(O)]—Y—, —Y—[C(O)NR^{c}]—, —Y—NR^{c}—Y—, —Y—[SiR^{c}₂]—Y—, a divalent C₁₋₄₀ alkyl group, a divalent C₂₋₄₀ alkenyl group, and a divalent C₁₋₄₀ haloalkyl group;
wherein:
Y, at each occurrence, independently is selected from a divalent C₁₋₄₀ alkyl group, a divalent C₂₋₄₀ alkenyl group, a divalent C₁₋₄₀ haloalkyl group, and a covalent bond;
R^{c} is selected from H, a C₁₋₆ alkyl group, a C₆₋₁₄ aryl group, and a —C₁₋₆ alkyl-C₆₋₁₄ aryl group; and
w is 0, 1, or 2; and
R^{a} is selected from a C₁₋₄₀ alkyl group, a C₂₋₄₀ alkenyl group, and a C₁₋₄₀ haloalkyl group, each of which optionally is substituted with 1-10 substituents independently selected from a halogen, —CN, NO₂, OH, —NH₂, —NH(C₁₋₂₀ alkyl), —N(C₁₋₂₀alkyl)₂, —S(O)₂OH, —CHO, —C(O)—C₁₋₂₀ alkyl, —C(O)OH, —C(O)—OC₁₋₂₀ alkyl, —C(O)NH₂, —C(O)NH—C₁₋₂₀ alkyl, —C(O)N(C₁₋₂₀ alkyl)₂, —OC₁₋₂₀ alkyl, —SiH₃, —SiH(C₁₋₂₀ alkyl)₂, —SiH₂(C₁₋₂₀ alkyl), and —Si(C₁₋₂₀ alkyl)₃.

19. The compound of claim 16, wherein R¹¹ and R¹² independently are selected from a linear or branched C₃₋₄₀ alkyl group, a linear or branched C₃₋₄₀ alkenyl group, —Y-phenyl and —Y-cyclohexyl, wherein Y is a divalent C₁₋₁₀ alkyl group or a covalent bond, and the phenyl group and the cyclohexyl group optionally are substituted with 1-5 substituents independently selected from a halogen, a C₁₋₆ alkyl group, a C₁₋₆ alkoxy group, and a C₁₋₆ haloalkyl group.

20. The compound of claim 16, wherein R¹¹ and R¹² independently are a linear or branched C₃₋₄₀ alkyl or C₃₋₄₀ alkenyl group.
